(19) United States
(12) Patent Application Publication
(10) Pub. No.: US 2013/0046134 A1
(43) Pub. Date: Feb. 21, 2013

(54) METHODS OF GENERATING ENGINEERED INNERVATED TISSUE AND USES THEREOF

(71) Applicants: Kevin Kit Parker, Waltham, MA (US); Adam W. Feinberg, Pittsburgh, PA (US); Lauren E.M. Chin, Champaign, IL (US)

(72) Inventors: Kevin Kit Parker, Waltham, MA (US); Adam W. Feinberg, Pittsburgh, PA (US); Lauren E.M. Chin, Champaign, IL (US)

(73) Assignee: President and Fellows of Harvard College, Cambridge, MA (US)

(21) Appl. No.: 13/580,191

(22) PCT Filed: Feb. 8, 2011
(86) PCT No.: PCT/US11/24029

§ 371 Date: Oct. 31, 2012

Related U.S. Application Data
(60) Provisional application No. 61/306,736, filed on Feb. 22, 2010.

(51) Int. Cl. A61N 1/05; A61F 2/04; C12Q 1/02; C12N 5/0793; G01N 33/566

(52) U.S. Cl. 600/36; 435/373; 435/7.21; 435/29; 607/129

(57) ABSTRACT

The present invention provides methods for generating relevant in vitro models of engineered innervated tissue, as well as uses of such tissues.

### RELATED APPLICATIONS

This application claims priority to U.S. Provisional Application No. 61/306,736, filed on Feb. 22, 2010, the entire contents of which are incorporated herein by this reference.

### BACKGROUND OF THE INVENTION

An estimated 14,000 neurons innervate the human heart to influence cardiac function (Armour, J. A., et al. (1997) The Anatomical Record 247: 289-298). The cardiac nervous system fine-tunes myocardial functions, such as fight-or-flight responses and stress responses by altering, e.g., myocardial contraction force, contraction rate, and conduction velocity. A disruption in the cardiac nervous system may contribute to atrial fibrillation, tachycardia, sudden cardiac death, and other arrhythmias as well as myocardial infarction and ischemia (Armour, J. A., et al. (2008) Experimental Physiology 93:165-176; Batulevicius, D., et al. (2008) Autonomic Neuroscience: Basic and Clinical 138:4-75; Cao, J. S., et al. (2000) Circulation Research 86: 816-821). Indeed, ischemic heart failure is the number one cause of death each year resulting in approximately 7.1 million deaths world-wide.

The cardiac nervous system, a part of the peripheral autonomic nervous system, consists of extrinsic and intrinsic networks of neurons (see, e.g., FIG. 15). Extrinsic neurons originate from outside of the heart, providing sympathetic and parasympathetic input to the heart from the brain via the spine. Sympathetic input stimulates cardiac function through adrenergic neurons increasing heart rate, conduction velocity and contraction force while parasympathetic input produces a reciprocal effect via cholinergic neurons (Armour, J. A., et al. (2008) Experimental Physiology 93:165-176). Intrinsic neurons exist around the heart itself, communicating with one another and cardiac cells in an intricate feedback loop. Recently, scientists have labeled the intrinsic cardiac nervous system the “little brain on the heart,” alluding to the system's role as a final coordinator of neural input, affecting local electrical and mechanical cardiac function (Armour, J. A., et al. (1997) The Anatomical Record 247: 289-298; Armour, J. A., et al. (2008) Experimental Physiology 93:165-176; Waldmann, M., et al. (2006) Journal of Applied Physiology 101:413-419).

The cardiac nervous system is a highly developed, complex network of inputs that all contribute to normal cardiac functioning and, ideally, any clinical manipulation of the heart must consider these complex effects. However, the current understanding of even basic neurocardiological problems is poor. For instance, after a heart transplant, heart rate variability is greatly reduced but, in some cases, gradually increases during months following the surgery. Autonomic re-innervation of the organ has been proposed as a reason for the gain in neurocardiac function, but understanding of the actual cause(s) remains insufficient for clinical application (Sanatani, S., et al. (2004) Pediatric Cardiology 25:114-118).

A more complex example surrounds sudden cardiac death which results in approximately 300,000 deaths per year.. A mounting body of evidence points to heterogeneous cardiac innervation post-infarction as a cause of tachycardia, heart rate over 100 beats per minute, and sudden cardiac death. To date, however, sudden cardiac death and other forms of arrhythmia such as ventricular arrhythmia and fibrillation have few clinical preventative treatments (Chen, L. S., et al. (2007) Journal of Cardiovascular Electrophysiology 18:123).

To consider these complex effects, an in vitro model of cultured cardiomyocytes is needed to study the heart in a controlled environment. To date, the standard of in vitro model of cardiac function is a neonatal rat cardiomycocyte model. This model offers the advantages of affordability, ease of use and housing, short gestation period, and each individual animal yields high cell numbers. Neonatal rat cardiomyocytes are also “cooperative cells”, as they readily adhere to a number of substrates and survive in culture for up to a week or more, unlike adult cardiomycocytes (Chlopcikova, S., et al. (2001) Biomedical Papers 145(2):49-55. Several groups have adapted the neonatal rat cardiomycocyte model to approximate innervated myocardium (Chen, L. S., et al. (2007) Journal of Cardiovascular Electrophysiology 18:12312- 14;1;. Horackova, M., et al. (1989) Canadian Journal of Physiology and Pharmacology 67:740-750; Ogawa, S., et al. (1992) Journal of Clinical Investigation 89: 1085-1093). These models vary widely, but all of them lack in vivo relevance as none attempt spatial organization of the tissue and none control individual cell architecture. Moreover, cardiomyocytes placed in an in vitro environment without external cues to guide their myofibrillar architecture, such as in existing models of innervated myocardium, lose their in vivo morphology and functionality.

Thus, there remains a need in the art for a more physiologically relevant in vitro model of innervated myocardium with relevant biological characteristics which can be easily produced in order to develop improved therapeutics, e.g., to treat ischemic heart disease.

### SUMMARY OF THE INVENTION

The present invention is based at least in part, on the discovery of methods for preparing an engineered innervated tissue. More specifically, it has been discovered that by co-culturing cells, such as myocytes (e.g., neonatal myocytes) with neurons, e.g., cortical neurons, on a solid support comprising a patterned biopolymer under appropriate conditions, an innervated tissue can be prepared. The methods described herein allow for the preparation of a more relevant in vitro model of engineered tissue in that the engineered tissue is innervated and displays properties of mature tissues, e.g., mature electrophysiology, such as mature action potential morphology, mature ion channel expression, and mature contractility, rather than the immature properties displayed by tissues/cells cultured using previously described methods.

Accordingly, in one aspect the present invention provides methods for preparing an engineered innervated tissue. The methods include providing a solid support structure comprising a patterned biopolymer, seeding immature cells on the patterned biopolymer, culturing the cells such that an anisotropic tissue forms, seeding the anisotropic tissue with neurons, and culturing the anisotropic tissue seeded with the neurons to form an anisotropic tissue with embedded neural networks, thereby preparing an engineered innervated tissue.

In another aspect, the present invention provides methods for accelerating maturation of a cultured cell. The methods include providing a solid support structure comprising a patterned biopolymer, seeding immature cells on the patterned biopolymer, culturing the cells such that an anisotropic tissue forms, seeding the anisotropic tissue with neurons, and culturing the anisotropic tissue seeded with the neurons to form an anisotropic tissue with embedded neural networks, thereby accelerating maturation of a cultured cell.

The biopolymer for use in the methods of the invention may be selected from the group consisting of extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, silks, and polyproteins. In one embodiment, the extracellular matrix protein is selected from the group consisting of fibronectin, vitronectin, laminin, collagen, fibrinogen, silk, and silk fibroin.

The biopolymer may be deposited on the solid support structure via soft lithography or printed on the solid support structure with a stamp, e.g., a polydimethylsiloxane stamp. In one embodiment, the methods of the invention further comprise printing multiple biopolymer structures, e.g., the same or different, with successive, stacked printings. The patterned biopolymer may include features with dimensions of about 5-40 micrometers.

The solid support for use in the methods of the invention may be a coverslip, a Petri dish or a multi-well plate, and in one embodiment, may further comprise a sacrificial polymer layer and a transitional polymer layer. In one embodiment, the sacrificial polymer is a degradable biopolymer. In one embodiment, the transitional polymer comprises polydimethylsiloxane.

In one embodiment, the immature cells are contractile cells. In one embodiment, the contractile cells are myocytes. In another embodiment, the contractile cells are glandular cells or smooth muscle cells. In another embodiment, the contractile cells are stem cells or progenitor cells. In one embodiment, the myocytes are cardiomyocytes. In one embodiment, the neuron is a neuron that does not secrete acetylcholine, epinephrine and/or norepinephrine. In another embodiment, the neurons are cortical neurons

In one embodiment, the neurons are seeded at a density of at least about 1.5×10⁶ per millimeter. In one embodiment, the myocytes are cultured for about 24 hours prior to the seeding of the neurons. In another embodiment, the myocytes are cultured for a period selected from the group consisting of about 2, 4, 6, 8, 10, 12, 14, 16, 18, 20, 22, 24, 26, 28, 30, 32, 34, 36, and 48 hours prior to the seeding of the neurons.

In one embodiment, the solid support structure comprises an optical signal capture device, and an image processing software to calculate a change in an optical signal.

In one embodiment, the methods of the invention further comprise stacking a plurality of tissues formed via the methods of the invention to produce a multi-layer tissue scaffold. In another embodiment, the methods of the invention further comprise growing the living cells in the tissue scaffold to produce three-dimensional, anisotropic myocardium. In another embodiment, the methods of the invention further comprise growing the living cells in the tissue scaffold to produce a replacement organ. In yet another embodiment, the methods of the invention further comprise wrapping the biopolymer around a three-dimensional implant and then inserting the implant into an organism.

In another aspect, the present invention provides methods for assaying a biological activity. The methods include providing an engineered innervated tissue prepared as described herein and evaluating an activity of the tissue, thereby assaying a biological activity.

Evaluating the biological activity may comprise evaluating the contractility of a cell, the mechano-electrical coupling of a cell, the mechano-chemical coupling of a cell, and/or the response of a cell to varying degrees of substrate rigidity.

In yet another aspect, the present invention provides methods for identifying a compound that modulates a tissue function. The methods include providing an engineered innervated tissue prepared as described herein, contacting the tissue with a test compound, and determining the effect of the test compound on a tissue function in the presence and absence of the test compound, wherein a modulation of the tissue function in the presence of the test compound as compared to the tissue function in the absence of the test compound indicates that the test compound modulates a tissue function, thereby identifying a compound that modulates a tissue function.

In another aspect, the present invention provides methods for identifying a compound useful for treating or preventing a tissue disease. The methods include providing an engineered innervated tissue prepared as described herein, contacting the tissue with a test compound, and determining the effect of the test compound on a tissue function in the presence and absence of the test compound, wherein a modulation of the tissue function in the presence of the test compound as compared to the tissue function in the absence of the test compound indicates that the test compound modulates a tissue function, thereby identifying a compound useful for treating or preventing a tissue disease.

In one embodiment, the tissue function is a biomechanical activity, such as contractility, cell stress, cell swelling, and rigidity. In one embodiment, the tissue function is an electrophysiological activity, such as action potential morphology, action potential duration, conduction velocity, calcium, e.g., Ca²⁺ ion, wave propagation velocity, calcium wave morphology, and change, e.g., increase or decrease relative to control, in calcium levels during systole and/or diastole.

In another aspect, the present invention provides methods for fabricating a pacemaker. The methods include providing a base layer, coating a sacrificial polymer layer on the base layer, coating a flexible polymer layer that is more flexible than the base layer on the sacrificial polymer layer, seeding cells on the flexible polymer layer, culturing the cells such that an anisotropic tissue forms, seeding the anisotropic tissue with neurons, culturing the anisotropic tissue seeded with the neurons to form an anisotropic tissue with embedded neural networks, and releasing the flexible polymer layer from the base layer to produce a pacemaker graft comprising the tissue structure, wherein the tissue structure is configured for epicardial attachment and is further configured to propagate an action potential through the attached tissue.

In one embodiment, the cells are derived from a sinoatrial node. In another embodiment, the cells are derived from an atrioventricular node.

In one embodiment, the methods further comprise harvesting the cells from a sinoatrial node. In another embodiment, the methods further comprise harvesting the cells from an atrioventricular node.

In one aspect, the present invention provides methods of treating a subject with a bradyarrythmia, comprising attaching the pacemaker prepared as described herein to the epicardium of the subject, thereby treating the subject with a bradyarrythmia.

In another aspect, the present invention provides methods of treating a subject with an AV-node conduction defect, comprising attaching the pacemaker prepared as described herein to the epicardium of the subject, such that the AV-node is bypassed.

### BRIEF DESCRIPTION OF THE DRAWINGS

FIG. 1 depicts representative action potentials from rat ventricular myocytes recorded at different points in development: 1 day (A), 5 days (B), 10 days (C) and as an adult (D). Figure obtained from Kilborn, M., Fedida, D. (1990) Journal of Physiology 430:37-60.

FIG. 2A depicts a schematic of an ion channel obtained from http://homepage.mac.comldtrapp/eChem.f/labB4.html; FIGS. 2B and 2c are schematics showing the ionic currents that contribute to the different parts of a day 1 old (B) or adult (C) rat ventricular action potential, adapted from Kilborn, M., Fedida, D. (1990) Journal of Physiology 430:37-60.

FIG. 3 depicts a schematic of soft lithography techniques (A) and micropatterning techniques (B) used to engineer cardiomyocytes into anisotropic monolayers.

FIG. 4 depicts engineered cardiac tissue stained against sarcomeric α-actinin, connexin 43, and DAPI. The culture exhibits a horizontal axis of anisotropy indicated by the white arrow as well as elongated, rod-like cell morphology. Scale: 20 μm.

FIG. 5 depicts the seeding order results. Seeding neurons 7 days before myocytes (A) created isotropic patterns. Seeding neurons 2 hours before seeding myocytes (B) produced poor cell adhesion of both cell types, and increased cell death (arrows). Seeding neurons 2 hours after seeding myocytes (C) produced good anisotropic pattern coverage and little cell death. Scale: 40 μm.

FIG. 6 depicts immunofluorescent images of a neural network stained against β-tubulin III. In the z-plane of the coverslip surface, the neurites may be seen (A). In a higher z the neurites are no longer in focus; the axons connecting the neurons in a network are in focus (B). Nuclei are DAPI-stained; myocytes are stained against sarcomeric α-actinin. Images obtained from Leica DM1 6000b. Scale: 10 μm.

FIG. 7 depicts neuron seeding concentrations of 3×10⁵ (A) and 1.5×10⁶ (B) formed networks in vitro. Images of neurons stained against β-tubulin III were obtained with a Leica DM1 6000b. Scale: 20 μm.

FIG. 8 depicts the results of staining each of the four co-culture conditions against sarcomeric α-actinin, β-tubulin III, and DAPI to label the myocytes, neurons, and all of the cells, respectively. (A). Scale: 10 μm. Using immunofluorescent microscopy, each nucleus was categorized as belonging to a neuron (solid arrows), myocyte (arrowheads) or other cell type (dashed arrow); the results are shown in B. The desired cell ratio, DCR (the ratio of neuron and myocytes to other cells) reflects the purity of the co-culture (C). The highest DCR existed in the HI 24 h co-culture.

FIG. 9 depicts an optical mapping system. The system employs 124 photodiode-coupled optical fibers. These fibers, when overlaid with a culture stained with a voltage-sensitive membrane dye, e.g., R11237, can interpret subtle changes in fluorescence corresponding directly to changes in transmembrane potential, maintaining both spatial and temporal information.

FIG. 10 depicts representative action potential (AP) morphologies from each of the co-cultures, and a day 4 myocyte only control. All co-culture AP's exhibit more rapid repolarization, visualized by a sharp point at the peak compared to a wide, triangle-like peak in the control myocyte AP. They also appear shorter in duration. The co-culture purity increases from left to right, corresponding to greater differences in AP morphology from control.

FIG. 11 is a graph quantifying the differing action potential morphologies observed in FIG. 10.

FIG. 12 depicts comparisons of action potential morphology and durations at day 5 during development and as an adult, adapted from (Kilborn, M., Fedida, D. (1990) Journal of Physiology 430:37-60) and that of the day 4 cardiomyocyte only control used herein and the HI 24 h co-culture. Scale bar: 200 ms.

FIG. 13 depicts action potential morphologies of day 5 myocytes in conditioned media are shown compared to day 5 control action potentials.

FIG. 14 are bar graphs indicating that no clear trends were noted in the measured conduction velocities of the co-cultures.

FIG. 15 depicts the peripheral autonomic nervous system and images of a cardiac myocyte and a neuron in vitro.

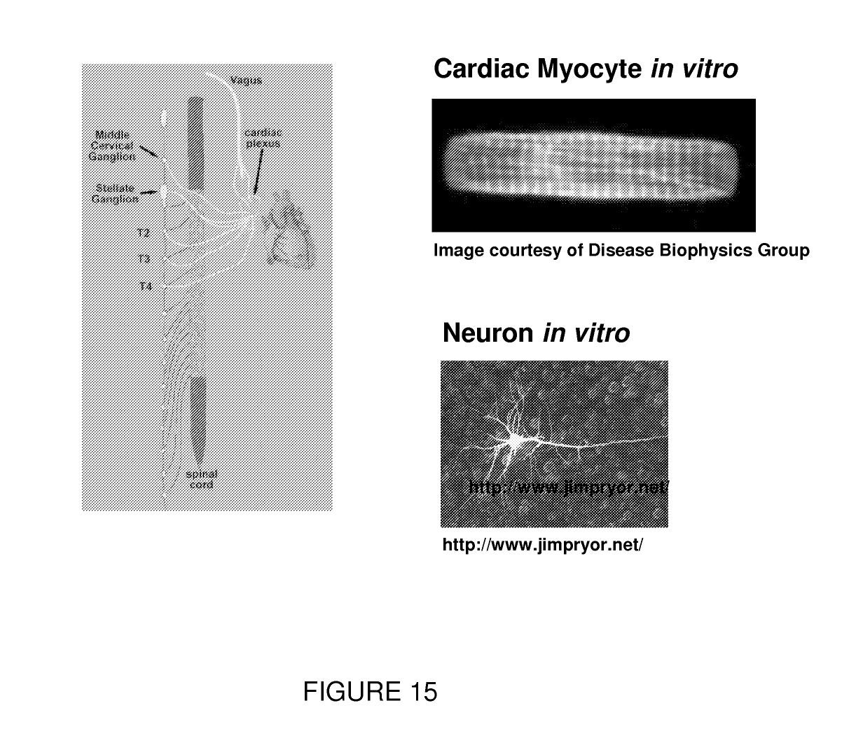

FIG. 16 depicts the identification of a biomodal co-culture distribution using the methods described herein.

FIG. 17 depcits the expected effects of ion channel blockers on the action porteinatial of the co-cultures described herein.

### DETAILED DESCRIPTION OF THE INVENTION

Described herein are improved methods for generating an engineered innervated tissue as well as methods for accelerating the maturation of cultured cells. Such methods for the preparation of engineered tissue, e.g., mature cardiac muscle tissue, with embedded neural networks, allow for the generation of an in vitro tissue model with a desirable combination of formerly mutually exclusive properties: (a) a tissue based on a cell source characterized by greater cell survivability and greater adhesion and (b) a tissue with in vivo relevance since the cells, e.g., cardiomyocytes, are engineered to achieve in vivo-like spatial organization both at the cellular- and tissue-scale and to include embedded neural networks which allow accelerated maturation of cellular and tissue properties, e.g., cardiac properties.

The engineered innervated tissues produced according to the methods of the invention may, for example, be used to study and/or to measure the contractility of cells with engineered shapes and connections, the mechano-electrical coupling of cells, the mechano-chemical coupling of cells, and/or the response of the cells to varying degrees of substrate rigidity. The engineered innervated tissues are also useful for investigating tissue developmental biology and disease pathology, as well as in drug discovery and toxicity testing. The methods of the invention may also be used to accelerate the maturation of cells, such as embryonic stem (ES) cells, or to prepare anisotropic muscle thin films (MTFs) which are electrically coupled and capable of transducing an action potential in vitro. Such anisotropic MTFs may be transplanted in vivo to successfully pace native heart tissue and/or allow conduction between cell populations, thus functioning as a pacemaker or as an AV bypass.

### I. Methods for Preparing Engineered Innervated Tissue

In one aspect, the present invention provides methods for preparing an engineered innervated tissue, as well as methods for accelerating maturation of a cultured cell. The methods include providing a solid support structure comprising a patterned biopolymer, seeding cells, e.g., contractile cells, e.g., immature contractile cells, such as neonatal cells, on the patterned biopolymer, culturing the cells under approriate conditions such that an anisotropic tissue forms, seeding the anisotropic tissue with neurons, and culturing the anisotropic tissue seeded with the neurons under approriate conditions such that an anisotropic tissue with embedded neural networks forms and/or such that maturation of a cultured cell is accelerated.

As used herein, the term “engineered innervated tissue” refers to a tissue prepared in accordance with the methods of the invention which displays at least one physical characteristic typical of the type of the tissue in vivo; and/or at least one functional characteristic typical of the type of the tissue in vivo, i.e., is functionally active; and is “innervated” (contains a neural network). For example, a physical characteristic of an engineered innervated muscle tissue may comprise the presence of parallel myofibrils with or without sarcomeres aligned in z-lines (which may be determined based upon, for example, microscopic examination). A functional characteristic of an engineered innervated muscle tissue may comprise an electrophysiological activity, such as an action potential, or biomechanical activity, such as contraction (which may be determined as described in, for example, U.S. Provisional Patent Application No. 61/174,511, PCT Patent Application Nos.: PCT/US09/45001, and PCT/US2010/033220 the entire contents of each of which are expressly incorporated herein by reference).

As used herein, the term “neural network” refers to a group(s), e.g., two or more, of chemically connected or functionally associated neurons (e.g., the neurons can transmit or transduce an electrical or chemical signal to another cell, such as a contractile cell, e.g., muscle cell, neural cell, glandular cell, or other cell type) in an engineered innervated tissue prepared according to the methods of the invention.

As used herein, the term “embedded” with respect to neural networks, refers to the direct contact of a portion, e.g., a neurite (e.g., an axon and/or dendrite) and/or a cell body, or an entire neural network with a cell, e.g., a contractile cell, e.g., a muscle cell, or tissue, or the insertion as an integral part of a portion or an entire neural network within a cell, e.g., a muscle cell, or tissue.

The term “anisotropic tissue”, as used herein refers to tissues whose properties (e.g., electrical conductivity and/or elasticity) are dependent on the direction in which the properties are measured. Examples of tissues which are anisotropic (e.g., in vivo) include muscle, collagen, skin, white matter, dentin, nerve bundles, tendon, ligament, and bone. Gor example, large nerves are anisotrpoic, with all of the nerve fibers running parallel to one another. In addition, an anisotropic muscle tissue may exhibit high electrical conductivity when such a measurement is conducted in one particular direction but not another, or may exhibit a mechanical activity (e.g., contractility and/or elasticity) when such a measurement is conducted in one particular direction but not another.

A. Preparing the Solid Support Structure Comprising a Paterned Biopolymer

The solid support structure for use in the methods of the invention comprises a patterned biopolymer which is applied to the solid support structure by, for example, microcontact printing of the biopolymer, using a stamp prepared by, for example, soft lithography, self assembly, vapor deposition or patterned photo cross-linking, as described in, for example WO 2008/045506, the contents of which are expressly incorporated herein by reference.

The solid support structure used in the methods of the invention may be formed of a rigid or semi-rigid material, such as a plastic, metal, ceramic, or a combination thereof. Suitable solid support structures for embodiments of the present invention include, for example, Petri dishes, cover-slips, or multi-well plates. The base layer may also be transparent, so as to facilitate observation. The support structure is ideally biologically inert, it has low friction with the tissues and it does not interact (e.g., chemically) with the tissues. Examples of materials that can be used to form the solid support structure include polystyrene, polycarbonate, polytetrafluoroethylene (PTFE), polyethylene terephthalate, quartz, silicon (e.g., silicon wafers) and glass. In one embodiment, the solid support structure layer is a silicon wafer, a glass cover slip, a multi-well plate or tissue culture plate.

In certain embodiments of the invention, the solid support structure is a multi-well, e.g., 12-, 24-, 48-, 96-well, plate and may further comprise an optical signal capture device and image processing software to calculate a change in optical signal, such as described in U.S. Provisional Patent Application No. 61/174,511 and PCT Patent Application No. PCT/US2010/033220, the entire contents of each of which are incorporated herein by reference. The optical signal capture device may further include fiber optic cables in contact with the culture wells.

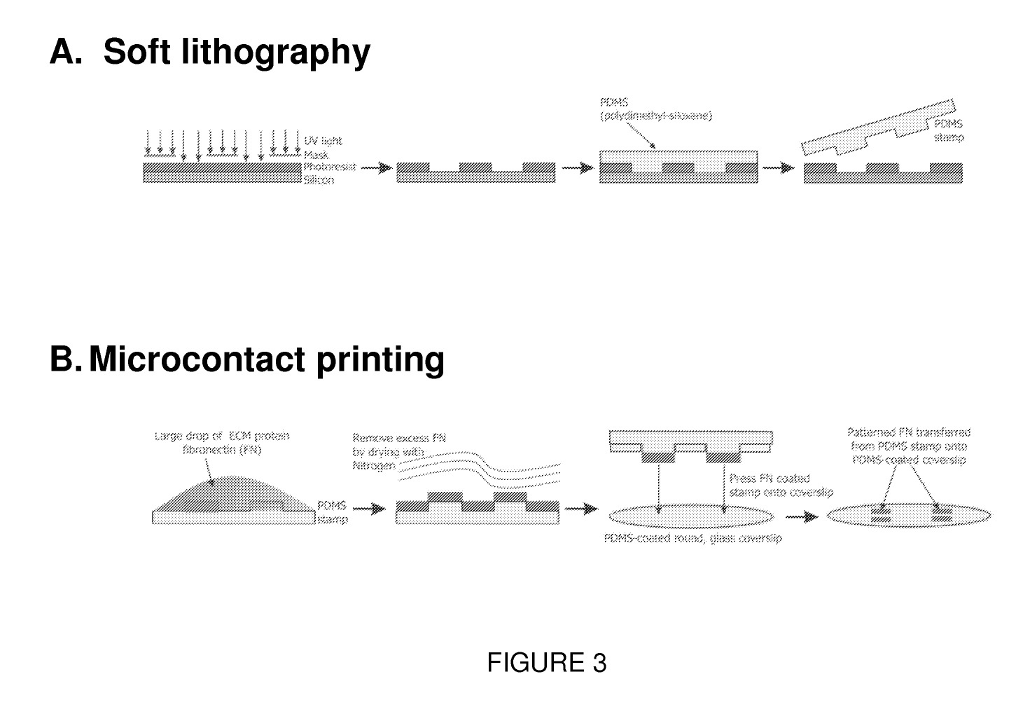

In order to prepare a solid support structure comprising a paterned biopolymer, a base layer is provided, and, for example, as depicted in FIG. 3, soft lithography may be used to prepare a stamp comprising any desired shape, e.g., a geometric shape, such as a square, circle, triangle, line, and combinations thereof, which is subsequently used to microcontact print a biopolymer on the base layer.

In order to prepare the stamp, a photoresist is deposited onto the base layer. To generate a pattern on the phostoresist, a solid mask, such as a photolithographic mask, is provided and placed on top of the photoresist layer. Subsequently, a portion of the photoresist layer (i.e., the portion of the photoresist not covered by the solid mask) is exposed to electromagnetic radiation. A suitable shape may be any desired shape, such as a geometric shape, e.g., a circle, square, rectangle, triangle, line, or combinations thereof. The mask, e.g., a micropatterned mask and/or a nanopatterned mask, placed on top of the photoresist layer is typically fabricated by standard photolithographic procedure, e.g., by means of electron beam lithography. The patterned stamp is prepared by depositing an elastomeric material on the base layer comprising the patterned photoresist. The patterned stamp is then used to print the biopolymer in the desired pattern on the solid support structure. In certain embodiments of the invention, an elastomeric substance may be deposited on the solid support structure.

“Biopolymer” refers to any proteins, carbohydrates, lipids, nucleic acids or combinations thereof, such as glycoproteins, glycolipids, or proteolipids.

Examples of suitable biopolymers that may be used for substrate include, without limitation:

(a) extracellular matrix proteins to direct cell adhesion and function (e.g., collagen, fibronectin, laminin, vitronectin, or polypeptides (containing, for example the well known -RGD- amino acid sequence));

(b) growth factors to direct specific cell type development cell (e.g., nerve growth factor, bone morphogenic proteins, or vascular endothelial growth factor);

(c) lipids, fatty acids and steroids (e.g., glycerides, non-glycerides, saturated and unsaturated fatty acids, cholesterol, corticosteroids, or sex steroids);

(d) sugars and other biologically active carbohydrates (e.g., monosaccharides, oligosaccharides, sucrose, glucose, or glycogen);

(e) combinations of carbohydrates, lipids and/or proteins, such as proteoglycans (protein cores with attached side chains of chondroitin sulfate, dermatan sulfate, heparin, heparan sulfate, and/or keratan sulfate); glycoproteins (selectins, immunoglobulins, hormones such as human chorionic gonadotropin, Alpha fetoprotein or Erythropoietin (EPO)); proteolipids (e.g., N-myristoylated, palmitoylated and prenylated proteins); and glycolipids (e.g., glycoglycerolipids, glycosphingolipids, or glycophosphatidylinositols);

(f) biologically derived homopolymers, such as polylactic and polyglycolic acids and poly-L-lysine;

(g) nucleic acids (e.g., DNA or RNA);

(h) hormones (e.g., anabolic steroids, sex hormones, insulin, or angiotensin);

(i) enzymes (e.g., oxidoreductases, transferases, hydrolases, lyases, isomerases, ligases; examples: trypsin, collegenases, or matrix metalloproteinases);

(j) pharmaceuticals (e.g., beta blockers, vasodilators, vasoconstrictors, pain relievers, gene therapy, viral vectors, or anti-inflammatories);

(k) cell surface ligands and receptors (e.g., integrins, selectins, or cadherins); and

(l) cytoskeletal filaments and/or motor proteins (e.g., intermediate filaments, microtubules, actin filaments, dynein, kinesin, or myosin).

In one embodiment, the biopolymer is fibronectin.

In another embodiment, the biopolymer is patterned as alternating high and low density lines, e.g., about 20-μm-wide lines. The biopolymer may include features with dimensions of about 1-15 micrometers, about 1-10, about 5-10, about 5-20, about 5-30, about 10-20, about 10-30, about 1-100, about 10-100, or about 20-100 micrometers. Dimensions and ranges intermediate to the above recited dimensions and ranges are also intended to be part of this invention.

In certain embodiments of the invention, the methods include printing multiple biopolymer structures with successive, stacked printings. For example, each biopolymer is different and the different proteins are printed in different (e.g., successive) printings. In another embodiment, each biopolymer is the same and printed in a different pattern in different (e.g., successive) printings.

In other embodiments of the invention, the biopolymer is constructed in a pattern such as a mesh or net structure which may produce a plurality of structures which may be stacked to produce a multi-layer tissue scaffold. Following construction of the biopolymer structure, living cells are integrated into or onto the scaffold to produce, e.g., a three-dimensional, anisotropic myocardium or other replacement organ (e.g., lung, liver, kidney, bladder). The methods of the invention may comprise a step of wrapping the biopolymer tissue structure around a three-dimensional implant and then inserting the implant into an organism.

In still other embodiments of the invention, the solid support structure may further comprise a sacrificial polymer layer and a transitional polymer layer, and the seeded cells may be cultured such that an innervated muscle thin film (MTF) is formed as similarly described in PCT Publication No. WO 2008/051265, the entire contents of which are incorporated herein by reference. Briefly, a solid support structure is coated with a sacrificial polymer layer; a flexible polymer layer is temporarily bonded to the solid support structure via the sacrificial polymer layer, and an engineered surface chemistry, e.g., a patterned biopolymer, is provided on the flexible polymer layer to enhance or inhibit cell and/or protein adhesion. Cells are seeded onto the flexible polymer layer, and co-cultured as described herein to form a tissue comprising, for example, patterned, innervated anisotropic myocardium.

In one embodiment, a desired shape of the flexible polymer layer can then be cut and the flexible film, including the polymer layer and tissue, can be peeled off with a pair of tweezers as the sacrificial polymer layer dissolves to release the flexible polymer layer, to produce a free-standing film, such as described in PCT Publication No. WO 2008/051265, and the horizontal and vertical MTFs described in U.S. Provisional Patent Application No. 61/174,511 and PCT Application No. PCT/US2010/033220, the entire contents of each of which are incorporated herein by reference.

B. Cells, Seeding, and Culturing

Cells, e.g., immature cells, such as stem cells, progenitor cells, induced pluripotent stem cells, embryonic cells, or neonatal cells, are seeded onto the patterned biopolymer and include, without limitation, such cells that will differentiate into muscle cells, skin cells, corneal cells, retinal cells, connective tissue cells, epithelial cells, glandular cells, endocrine cells, adipose cells, and lymphatic cells, or combinations of such cells. One of ordinary skill in the art may readily distinguish an immature cell from a mature cell using routine techniques (e.g., histological, biomechanical, electrophysiological techniques), such as those described below. As used herein, muscle cells include smooth muscle cells, striated muscle cells (skeletal), or cardiac cells. Stem cells including embryonic (primary and cell lines), fetal (primary and cell lines), adult (primary and cell lines) and iPS (induced pluripotent stem cells) may be used.

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “progenitor cell” is used herein synonymously with “stem cell.”

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation”.

The term “embryonic stem cell” is used to refer to the pluripotent stem cells of the inner cell mass of the embryonic blastocyst (see U.S. Pat. Nos. 5,843,780, 6,200,806, the contents of which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non- embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells.

In one embodiment, progenitor cells suitable for use in the claimed methods are Committed Ventricular Progenitor (CVP) cells as described in PCT Application No. PCT/US09/060224, entitled “Tissue Engineered Mycocardium and Methods of Productions and Uses Thereof”, filed October 9, 2009, the entire contents of which are incorporated herein by reference.

Cells may be normal cells or abnormal cells (e.g., those derived from a diseased tissue, or those that are physically or genetically altered to achieve a abnormal or pathological phenotype or function), normal or diseased cells derived from embryonic stem cells or induced pluripotent stem cells, or normal cells that are seeded/printed in an abnormal or aberrant configuration. In one embodiment, the cells for use in the methods of the invention, although derived from a tissue, comprise a single cell type. In one embodiment, the cells for use in the claimed methods are myocytes.

In certain embodiments of the invention, the cells may be cells derived from a sinoatrial or an atrioventricular node. In other embodiments of the invention, the cells may be genetically altered such that they possess the electrical excitation or pacemaker properties necessary for biological pacemaker or AV-node bypass function. In some embodiments, the cells are genetically engineered to express an ion channel that promotes pacemaking and/or electrical excitability. Suitable ion channels include, but are not limited to, hyperpolarisation-activated cyclic nucleotide-gated (HCN) channels, e.g., HCN1, HCN2, HCN3, or HCN4. Such ion channels are encoded by an HCN gene, e.g., a human HCN gene. Suitable adult mesenchymal stem cells expressing an HCN are described in WO2008011134 and Plotnikov et al., Circulation, 2007, 116(7):706-713, the entire contents of which are hereby incorporated herein by reference. In other embodiments, cells are genetically engineered to give them stem-cell characteristics such that they can be subsequently differentiated into a cell type which possesses the electrical excitation or pacemaker properties necessary for biological pacemaker or AV-node bypass function. Cells from any species can be used so long as they do not cause an adverse immune reaction in the recipient.

To seed cells, solid support structures comprising a patterned biopolymer (optionally comprising a sacrificial polymer layer and/or a transitional polymer layer) are placed in culture with a cell suspension allowing the cells to settle and adhere to the patterned biopolymer. The cells on the solid support structures may be cultured in an incubator under physiologic conditions (e.g., at 37° C.) until the cells form a two-dimensional (2D) tissue (e.g., a layer of cells that is less than 200 microns thick, or, in particular embodiments, less than 100 microns thick, or even just a monolayer of cells), the anisotropy of which is determined by the engineered surface chemistry.

One of ordinary skill in the art may readily determine appropriate seeding concentrations and culture times suitable for the formation of a desired anisotropic tissue. For example, cells, such as myocytes, may be seeded at any appropriate density, such as about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶. In one embodiment of the invention, cells are seeded at a density of about 1.5×10⁶. Amounts intermediate to the above recited amounts are also intended to be part of this invention.

For the formation of an anisotropic tissue, the cells may be cultured for about 0.25 hours, about 0.5 hours, about 0.75 hours, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4, about 4.25 hours, about 4.5 hours, about 4.75 hours, about 5 hours, about 5.25 hours, about 5.5 hours, about 5.75 hours, about 6 hours, about 6.25 hours, about 6.5 hours, about 6.75 hours, about 7 hours, about 7.25 hours, about 7.5 hours, about 7.75 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, or about 48 hours prior to the seeding of neurons. Times intermediate to the above recited times are also intended to be part of the invention.

The determination of whether the cells have formed an anisotropic tissue is well within the level of ordinary skill in the art and may be based on microscopic examination of the cultures, staining for molecules associated with the desired tissue, determination of gene expression or protein production of molecules associated with the desired tissue, and/or assessment of a biomechanical and/or electrophysiological activity associated with the desired tissue. For example, as described in the Examples section below, determining whether cultured myocytes have formed an anisotrpic tissue may comprise microscopic examination and immunohistochemical analysis for, e.g., myosin, myoglobin, and atrial natriuretic peptide (ANP).

Suitable culture media may be determined by one of ordinary skill in the art and will contain any nutrients suitable to promote growth and sustain the survival of the cells and neurons, such as serum, amino acids, vitamins, minerals, and may further comprise an antibiotic(s) and/or a suitable growth factor(s), such as nerve growth factor.

Neurons may be seeded on the anisotropic tissue by placing a cell suspension of the neurons in culture with the anisotropic tissue at the appropriate time frame (see above) and culturing in an incubator under physiologic conditions (e.g., at 37° C.). The neurons may be seeded at any appropriate density, such at 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶. In one embodiment of the invention, the neurons are seeded at a density of about 1.5×10⁶. Amounts intermediate to the above recited amounts are also intended to be part of this invention.

One of ordinary skill in the art may determine appropriate densities of cells and/or neurons to seed based on methods routine in the art and described herein. For example, as demonstrated in the Examples section below, neurons (and other cell types) isolated from a tissue may contain the desired cell type (e.g., neurons) as well as other, undesired cell types (e.g., glial cells and fibroblasts). Thus, the density of cells and/or neurons seeded may be varied in order to optimize the percentage of desired cell type(s) relative to undesired cell type(s) in the co-culture. The co-cultures may comprise a population of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90% of the desired cell type(s) and/or neuron relative to an undesired cell type(s).

The co-cultures may also be optimized such that the co-cultures comprise populations of undesired cells of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30% about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, or about 49%, relative to the desired cell type(s) and/or neuron.

Once seeded, the co-cultures may be cultured for a sufficient time and under appropriate conditions to form an anisotropic tissue with embedded neural networks. For example, the co-cultures may be cultured for about 24 hours, about 30 hours, about 31 hours, about 32 hours, about 33 hours, about 34 hours, about 35, hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 47 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, about 80 hours, about 81 hours, about 82 hours, about 83 hours, about 84 hours, about 85 hours, about 86 hours, about 87 hours, about 88 hours, about 89 hours, about 90 hours, about 91 hours, about 92 hours, about 93 hours, about 94 hours, about 95 hours, about 96 hours, about 97 hours, about 98 hours, about 99 hours, about 100 hours, about 101 hours, about 102 hours, about 103 hours, about 104 hours, about 105 hours, about 106 hours, about 107 hours, about 108 hours, about 109 hours, about 110 hours, about 111 hours, about 112 hours, about 113 hours, about 114 hours, about 115 hours, about 116 hours, about 117 hours, about 118 hours, about 119 hours, about 120 hours, about 121 hours, about 122 hours, about 123 hours, about 124 hours, about 125 hours, about 126 hours, about 127 hours, about 128 hours, about 129 hours, about 130 hours, about 131 hours, about 132 hours, about 133 hours, about 134 hours, about 135 hours, about 136 hours, about 137 hours, about 138, about 139 hours, about 140 hours, about 141 hours, about 142 hours, about 143 hours, about 144 hours, about 145 hours, about 146 hours, about 147 hours, about 148 hours, about 149 hours, about 150 hours, about 151 hours, about 152 hours, about 153 hours, about 154 hours, about 155 hours, about 156 hours, about 157 hours, about 158 hours, about 159 hours, about 160 hours, about 161 hours, about 162 hours, about 163 hours, about 164 hours, about 165 hours, about 166 hours, about 167 hours, or about 168 hours to form an anisotropic tissue with embedded neural networks . Times intermediate to the above recited times are also intended to be part of this invention.

Neurons for use in the claimed methods are preferably primary neurons and may be derived from any suitable source such as E16-E18 fetal rats, E15-E16 fetal mice, neonatal rats, or neonatal mice. Neurons from other animal species may be obtained from developmentally equivalent time points. Human neurons may be differentiated from human embryonic stem cells. Additionally, induced pluripotent stem cells derived from human somatic cells, e.g., fibroblasts, can be differentiated into neurons. Alternatively, ES cells, such as neural stem cells, from e.g., fetal spinal cord, may be cultured to form neurons. Suitable neurons may be sympathetic neurons, parasympathetic neurons, or cortical neurons. In certain embodiments of the invention, the neuron is a neuron that does not secrete acetylcholine, epinephrine, and/or norepinephrine. In other embodiments of the invention, the neuron is a cortical neuron. Methods for the isolation and culturing of cortical, parasympathetic, and sympathetic neurons are known in the art and described in, for example, “New Methods for Culturing Cells From Neurons”, Vol. 1. 2005. P. Poindron, P. Piguet, and E. Forster, eds. BioValley Monographs, Basel, Karger, pp.12-22; Meberg, P. J. and M. W. Miller. (2003) Methods Cell Biol 71:111; Whitfield, et al. (2002) in Methods in Molecular Biol Humana Press, p.157.

In certain embodiments of the invention, cell explants are prepared and neurons for seeding the anisotropic tissue are isolated from such explants. In other embodiments of the invention, primary neurons are pre-plated and/or filtered to select against undesired cell types.

In one embodiment, myocytes are cultured to form an anisotropic tissue, and co-cultured with neurons.

The determination of whether an engineered innervated tissue has been prepared and/or whether the tissue has matured may be based on microscopic examination of the tissue and/or expression analysis of the tissue and/or electrophysiological activity of the tissue and/or biomechanical activity of the tissue. As described in detail in the Examples section below, the co-culture methods generate a tissue with ion channel expression, action potential morphology, and contractility typical of in vivo adult cells, and such parameters may be measured to determine maturity and/or innervation of the tissue. For example, co-cultures which contain living cells that stain with, e.g., β-tubulin III, atrial naturitic peptide, Sca-1, myosin, adrenergic receptors and/or muscarinic receptors, display rapid repolarization and short action potential durations which may be assessed by determining, e.g., action potential durations as compared to co-cultures that are not innervated and/or immature are determined to be matured and/or innervated.

### II. Uses of the Engineered Innervated Tissues

The engineered innervated tissues produced according to the methods of the invention may be used in various applications e.g., to measure various biological activities or functions, such as the contractility of tissues with engineered shapes and connections, the mechano-electrical coupling of tissues, the mechano-chemical coupling of tissues, and/or the response of the tissues to varying degrees of substrate rigidity.

Biological activities or functions that can be measured include, e.g., biomechanical forces that result from stimuli that include, but are not limited to, cell/tissue contraction, osmotic swelling, structural remodeling and tissue level pre-stress, and electrophysiological responses, in a non-invasive manner, for example, in a manner that avoids cell/tissue damage, and in an manner replicating an in vivo environment. Exemplary assays are disclosed herein and in PCT Application No. PCT/US2010/033220, the contents oh which are incorporated herein in their entirety.

Accordingly, the present invention provides methods for assaying a biological activity. The methods include providing engineered innervated tissue prepared according to the methods of the invention and evaluating an activity of the tissue, such as a biomechanical or electrophysiological activity. The methods may include evaluating a biomechanical or electrophysiological activity at one time point or more than one time point.

In one embodiment, such an assay may be used to evaluate the contractility of engineered innervated tissues. This assay evaluates the contraction response of the tissue when exposed to varying stimuli. In another embodiment, such an assay may be used to evaluate the mechanical communication between cells (cell-cell) or between cell and extracellular matrix (cell-matrix). Such an assay could evaluate the relationship between cellular shape, orientation, or distance to the cell-cell or cell-matrix communication. In another embodiment, such an assay may be used to evaluate the mechanics of a cell's nucleus. The effect of varying substrate rigidity on tissue structure and function may also be evaluated using the assays of the invention. Other assays include mechano-electrical or mechano-chemical coupling of cells/tissues, varying cell shape and connection.

The assays of the present invention may further comprise, e.g., imaging the tissues and/or staining the tissues for a particular cell type or gene or protein expression.

The engineered innervated tissues of the present invention are also useful for investigating tissue developmental biology and disease pathology, as well as in drug discovery and toxicity testing.

Accordingly, the present invention also provides methods for identifying a compound that modulates a tissue function. The methods include contacting an engineered innervated tissue prepared as described herein with a test compound; and determining the effect of the test compound on a tissue function in the presence and absence of the test compound, wherein a modulation of the tissue function in the presence of said test compound as compared to the tissue function in the absence of said test compound indicates that said test compound modulates a tissue function, thereby identifying a compound that modulates a tissue function.

In another aspect, the present invention provides methods for identifying a compound useful for treating or preventing a disease. The methods include contacting an engineered innervated tissue prepared as described herein with a test compound; and determining the effect of the test compound on a tissue function in the presence and absence of the test compound, wherein a modulation of the tissue function in the presence of the test the as compared to the tissue function in the absence of the test compound indicates that the test compound modulates a tissue function, thereby identifying a compound useful for treating or preventing a disease.

The methods of the invention generally comprise determining the effect of a test compound on a plurality of cells or cell types, e.g., a tissue, however, the methods of the invention may comprise further evaluating the effect of a test compound on an individual cell type(s).

The present invention also includes the production of arrays. Arrays of engineered innervated tissue prepared according to the methods described herein may be prepared using, for example, multi-well plates or tissue culture dishes (as described in, for example, PCT Application No. PCT/US2010/033220 the contents oh which are incorporated herein in their entirety) so that each engineered innervated tissue in a particular well may be exposed to a different compound to investigate the effect of the compound on the tissue (e.g., altered expression of a given protein/cell surface marker, or altered differentiation). Thus, the screening methods of the invention may involve contacting a single tissue with a test compound or a plurality of tissues with a test compound.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (e.g., contacting a cell or tissue prepared according to the methods described herein with a test compound) is intended to include any form of interaction (e.g., direct or indirect interaction) of a test compound and a cell or tissue. The term contacting includes incubating a compound and a cell or tissue (e.g., adding the test compound to a cell or tissue).

Test compounds, may be any agents including chemical agents (such as toxins), small molecules, pharmaceuticals, peptides, proteins (such as antibodies, cytokines, enzymes, and the like), and nucleic acids, including gene medicines and introduced genes, which may encode therapeutic agents, such as proteins, antisense agents (i.e., nucleic acids comprising a sequence complementary to a target RNA expressed in a target cell type, such as RNAi or siRNA), ribozymes, and the like.

The test compound may be added to a cell or tissue by any suitable means. For example, the test compound may be added drop-wise onto the surface of a cell or tissue on and allowed to diffuse into or otherwise enter the cell or tissue, or it can be added to the nutrient medium and allowed to diffuse through the medium. In the embodiment where the cell or tissue is cultured in a multi-well plate, each of the culture wells may be contacted with a different test compound or the same test compound. In one embodiment, the screening platform includes a microfluidics handling system to deliver a test compound and simulate exposure of the microvasculature to drug delivery.

Numerous physiologically relevant parameters, such as insulin secretion, conductivity, neurotransmitter release, lipid production, bile secretion, biomechanical and electrophysiological activities, action potential morphologies, action potential duration , conduction velocity, can be evaluated in the methods of the invention. For example, in one embodiment, engineered innervated tissue prepared according to the methods described herein can be used in contractility assays for muscular cells or tissues, such as chemically and/or electrically stimulated contraction of vascular, airway or gut smooth muscle, cardiac muscle or skeletal muscle. In addition, the differential contractility of different muscle cell types to the same stimulus (e.g., pharmacological and/or electrical) can be studied.

In another embodiment, engineered innervated tissue prepared according to the methods described herein can be used for measurements of solid stress due to osmotic swelling of cells. For example, as the cells swell the soft substrate will deform and as a result, volume changes, force and points of rupture due to cell swelling can be measured.

In another embodiment, engineered innervated tissue prepared according to the methods described herein can be used for pre-stress or residual stress measurements in cells. For example, vascular smooth muscle cell remodeling due to long term contraction in the presence of endothelin-1 can be studied.

Further still, engineered innervated tissue prepared according to the methods described herein can be used to study the loss of rigidity in tissue structure after traumatic injury, e.g., traumatic brain injury. Traumatic stress can be applied to vascular smooth muscle engineered innervated tissues as a model of vasospasm. These engineered innervated tissues can be used to determine what forces are necessary to cause vascular smooth muscle to enter a hyper-contracted state. These engineered innervated tissues can also be used to test drugs suitable for minimizing vasospasm response or improving post-injury response and returning vascular smooth muscle contractility to normal levels more rapidly.

In other embodiments, engineered innervated tissue prepared according to the methods described herein can be used to study biomechanical responses to paracrine released factors (e.g., vascular smooth muscle dilation due to release of nitric oxide from vascular endothelial cells, or cardiac myocyte dilation due to release of nitric oxide).

In other embodiments, engineered innervated tissue prepared according to the methods described herein can be used to evaluate the effects of a test compound on an electrophysiological parameter, e.g., an electrophysiological profile comprising a voltage parameter selected from the group consisting of action potential, action potential duration (APD), conduction velocity (CV), refractory period, wavelength, restitution, bradycardia, tachycardia, reentrant arrhythmia, and/or a calcium flux parameter, e.g., intracellular calcium transient, transient amplitude, rise time (contraction), decay time (relaxation), total area under the transient (force), restitution, focal and spontaneous calcium release. For example, a decrease in a voltage or calcium flux parameter of a engineered innervated tissue comprising cardiomyocytes upon contacting the engineered innervated tissue with a test compound, would be an indication that the test compound is cardiotoxic.

In yet another embodiment, engineered innervated tissue prepared according to the methods described herein can be used in pharmacological assays for measuring the effect of a test compound on the stress state of a tissue. For example, the assays may involve determining the effect of a drug on tissue stress and structural remodeling of engineered innervated tissues. In addition, the assays may involve determining the effect of a drug on cytoskeletal structure and, thus, the contractility of the engineered innervated tissues.

In still other embodiments, engineered innervated tissue prepared according to the methods described herein can be used to measure the influence of biomaterials on a biomechanical response. For example, differential contraction of vascular smooth muscle remodeling due to variation in material properties (e.g., stiffness, surface topography, surface chemistry or geometric patterning) of engineered innervated tissues can be studied.

In further embodiments, engineered innervated tissue prepared according to the methods described herein can be used to study functional differentiation of immature, e.g., stem cells (e.g., pluripotent stem cells, multipotent stem cells, induced pluripotent stem cells, and progenitor cells of embryonic, fetal, neonatal, juvenile and adult origin) into contractile phenotypes. For example, undifferentiated cells are seeded on a patterned biopolymer, e.g., immature cells, and differentiation into a contractile phenotype is observed by evaluating biopolymer displacement. Differentiation can be observed as a function of: action potential morphology, action potential duration, conduction velocity, co-culture (e.g., co-culture with differentiated cells), paracrine signaling, pharmacology, electrical stimulation, magnetic stimulation, thermal fluctuation, transfection with specific genes and biomechanical perturbation (e.g., cyclic and/or static strains)

In another embodiment, engineered innervated tissue prepared according to the methods described herein may be used to determine the toxicity of a test compound by evaluating, e.g., the effect of the compound on an electrophysiological response of a engineered innervated tissue. For example, opening of calcium channels results in influx of calcium ions into the cell, which plays an important role in excitation-contraction coupling in cardiac and skeletal muscle fibers. The reversal potential for calcium is positive, so calcium current is almost always inward, resulting in an action potential plateau in many excitable cells. These channels are the target of therapeutic intervention, e.g., calcium channel blocker sub-type of anti-hypertensive drugs. Candidate drugs may be tested in the electrophysiological characterization assays described herein to identify those compounds that may potentially cause adverse clinical effects, e.g., unacceptable changes in cardiac excitation, that may lead to arrhythmia.

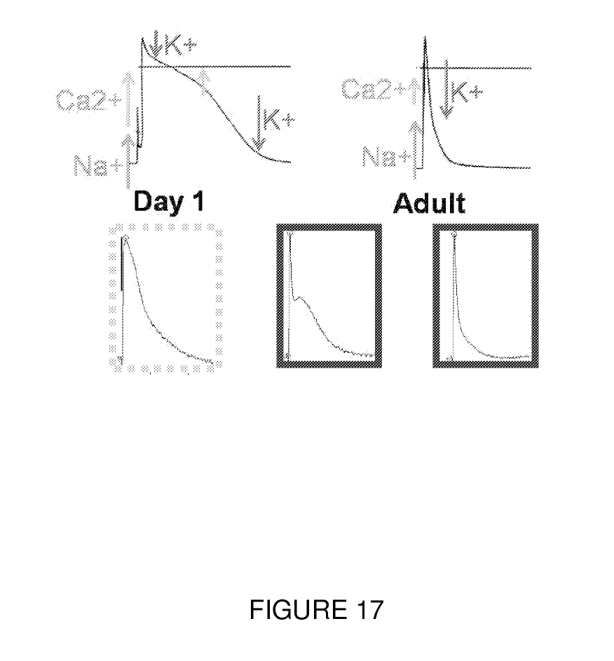

For example, unacceptable changes in cardiac excitation that may lead to arrhythmia include, e.g., blockage of ion channel requisite for normal action potential conduction, e.g., a drug that blocks Na⁺channel, e.g., Tetrotodoxin, would block the action potential and no upstroke would be visible; a drug that blocks Ca²⁺ channels, e.g., Nifedipine, would prolong repolarization and increase the refractory period; blockage of K⁺ channels would block rapid repolarization, and, thus, would be dominated by slower Ca²⁺ channel mediated repolarization (see, e.g., FIG. 17).

In addition, metabolic changes may be assessed to determine whether a test compound is toxic by determining, e.g., whether contacting a engineered innervated tissue with a test compound results in a decrease in metabolic activity and/or cell death. For example, detection of metabolic changes may be measured using a variety of detectable label systems such as fluormetric/chrmogenic detection or detection of bioluminescence using, e.g., AlamarBlue fluorescent/chromogenic determination of REDOX activity (Invitrogen), REDOX indicator changes from oxidized (non-fluorescent, blue) state to reduced state(fluorescent, red) in metabolically active cells; Vybrant MTT chromogenic determination of metabolic activity (Invitrogen), water soluble MTT reduced to insoluble formazan in metabolically active cells; and Cyquant NF fluorescent measurement of cellular DNA content (Invitrogen), fluorescent DNA dye enters cell with assistance from permeation agent and binds nuclear chromatin. For bioluminescent assays, the following exemplary reagents is used: Cell-Titer Glo luciferase-based ATP measurement (Promega), a thermally stable firefly luciferase glows in the presence of soluble ATP released from metabolically active cells.

Engineered innervated tissue prepared according to the methods described herein is also useful for evaluating the effects of particular delivery vehicles for therapeutic agents e.g., to compare the effects of the same agent administered via different delivery systems, or simply to assess whether a delivery vehicle itself (e.g., a viral vector or a liposome) is capable of affecting the biological activity of the engineered innervated tissue. These delivery vehicles may be of any form, from conventional pharmaceutical formulations, to gene delivery vehicles. For example, engineered innervated tissue prepared according to the methods described herein may be used to compare the therapeutic effect of the same agent administered by two or more different delivery systems (e.g., a depot formulation and a controlled release formulation). Engineered innervated tissue prepared according to the methods described herein may also be used to investigate whether a particular vehicle may have effects of itself on the tissue. As the use of gene-based therapeutics increases, the safety issues associated with the various possible delivery systems become increasingly important. Thus, the engineered innervated tissue prepared according to the methods described herein may be used to investigate the properties of delivery systems for nucleic acid therapeutics, such as naked DNA or RNA, viral vectors (e.g., retroviral or adenoviral vectors), liposomes and the like. Thus, the test compound may be a delivery vehicle of any appropriate type with or without any associated therapeutic agent.

Furthermore, engineered innervated tissue prepared according to the methods described herein is a suitable in vitro model for evaluation of test compounds for therapeutic activity with respect to, e.g., a muscular and/or neuromuscular disease or disorder. For example, the engineered innervated tissue prepared according to the methods described herein may be contacted with a candidate compound by, e.g., immersion in a bath of media containing the test compound, and the effect of the test compound on a tissue activity (e.g., a biomechanical and/or electrophysiological activity) may measured as described herein, as compared to an appropriate control, e.g., an untreated engineered innervated tissue. Alternatively, an engineered innervated tissue prepared according to the methods described herein may be bathed in a medium containing a candidate compound, and then the tissues are washed, prior to measuring a tissue activity (e.g., a biomechanical and/or electrophysiological activity) as described herein. Any alteration to an activity determined using the engineered innervated tissue in the presence of the test agent (as compared to the same activity using the device in the absence of the test compound) is an indication that the test compound may be useful for treating or preventing a tissue disease, e.g., a neuromuscular or cardiac disease.

As described above, certain embodiments of the invention allow for the formation of an innervated muscle thin film. Such anisotropic muscle thin films (MTFs) are electrically coupled and capable of transducing an action potential in vitro and may be transplanted in vivo to successfully pace native heart tissue and/or allow conduction between cell populations, thus functioning as a pacemaker or as an AV bypass.

Accordingly, in one embodiment, the invention provides methods of fabricating a pacemaker as described in detail in U.S. Provisional Patent Application Nos. 61/249,870, filed on Oct. 8, 2009 and 61/391,203 filed on Oct. 8, 2010. Such a pacemaker may be used to treat a subject with a bradyarrythmia or a subject with an AV-node conduction defect. Any suitable means for accessing the heart tissue and implanting the pacemaker into the heart may be used including, but not limited to, e.g., thoracic surgery or transmyocardial catheter delivery. In some embodiments, a pacemaker is rolled up inside a transmyocardial catheter prior to implantation and subsequently unrolled when the site of implantation is reached. A temporary force may be applied to the pacemaker upon implanting the pacemaker graft in vivo to hold the graft onto the host until cellular junctions are established, thereby connecting the pacemaker with cells of the host tissue.

The exact size and shape of the pacemaker may readily be determined by one of ordinary skill in the art. For example, an adult human may require a pacemaker that is typically 2-4 cm in length for a square or rectangular shape or 3 cm in diameter for a circular shape. The size of the pacemaker graft can be designed according to the needs of the patient. Suitable surface areas for the pacemaker grafts include, but are not limited to, e.g., 1 to 10⁶ mm², 10 to 10⁵ mm², 10² to 10⁴ mm², or, 10² to 10³ mm². Suitable lengths for pacemaker grafts include, but are not limited to, e.g., 0.1, 05, 1, 2, 3, 4, 5, 6, 7, 8, 9, 10, 11, 12, 13, 14, 15, 16, 17, 18, 19, 20, 25, 30, 40, 50 or 100 cm. Patients with hypertrophic hearts may require larger pacemaker grafts than those with normal sized hearts. Likewise, pediatric patients may require smaller pacemaker grafts than adult patients.

The shape of the pacemaker can be designed according to the needs of the patient. The overall shape of the pacemaker is optimized to possess desirable biological properties, and to efficiently deliver depolarizing current to the host myocardium with as few pacemaking cells as possible. For example, the shape of a pacemaker can be designed to be elliptical, to deliver a directional, polarizing current to the surrounding cardiac tissue, to allow for tuning the direction of wavefront propagation. The incorporation of non-excitable cells (cardiac fibroblasts, for example) may also be used to block propagation in one direction in order to deliver more depolarizing current in the opposite direction.

This invention is further illustrated by the following examples which should not be construed as limiting. The contents of all references, patents and published patent applications cited throughout this application, as well as the Figures, are hereby incorporated herein by reference.

### EXAMPLES

### Example 1

### Engineered Cardiac Muscle

### Introduction

The present invention provides improved in vitro models of innervated tissue, e.g., myocardium. The in vitro models described herein 1) spatially organize neonatal rat ventricular cardiomyocytes to create an in vivo-like monolayer of aligned, rod-shaped cells, 2) provide optimized co-culture conditions to best embed the neurons into networks that can functionally affect the myocyte monolayer, and 3) accelerate maturation of the cardiomyocyte electrophysiology by the addition of the neural networks.

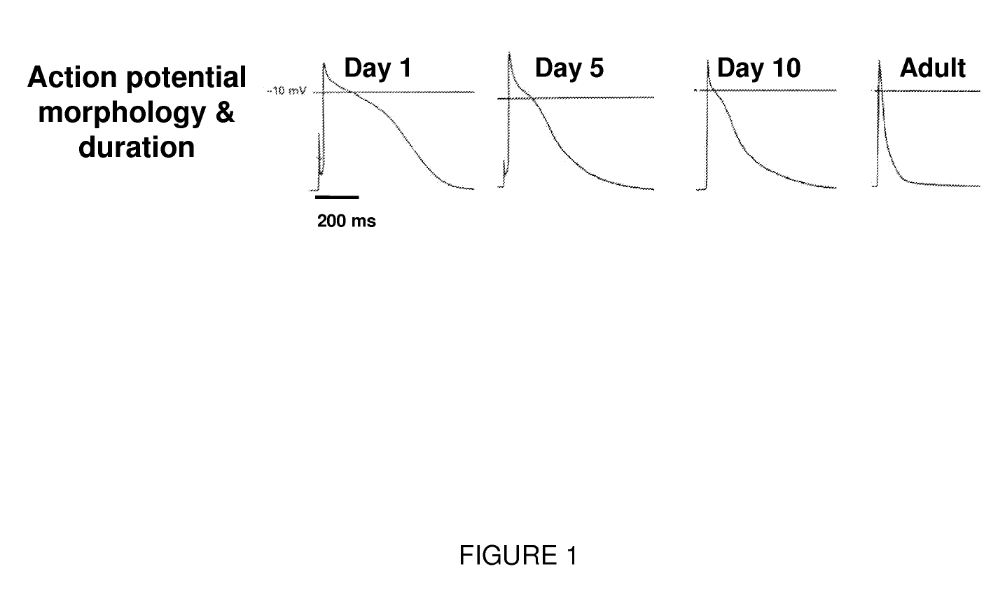

Rat cardiac electrophysiology changes drastically during development. Throughout neonatal development, rat cardiac action potentials change in both morphology and duration. FIG. 1 shows typical action potential recordings from rat ventricular myocytes recorded at different points during neonatal development. As seen in FIG. 1, the slope of the action potential just after the peak becomes steeper during development, and the shape of the triangle at the peak becomes narrower. This corresponds to more rapid repolarization just after peak depolarization. The action potential also becomes much shorter in duration during development.

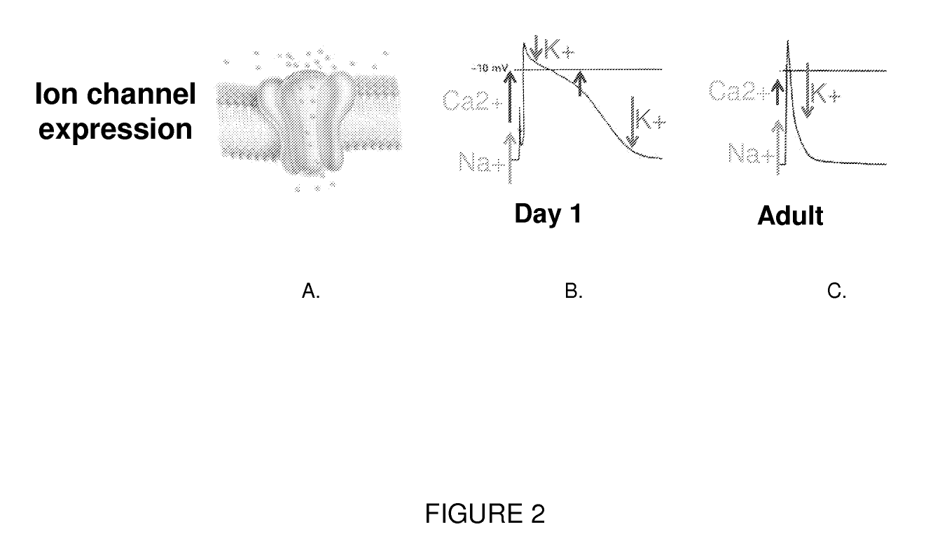

Underlying the changes in action potential morphology and duration over time are changes in ion channel expression. FIG. 2A shows a schematic of an open ion channel. Small ionic molecules are able to cross through the membrane via this pore. FIGS. 2B and 2C show the various ionic currents that dominate various parts of the rat cardiac action potential at day 1 in development (immature) and at the adult stage. In both immature and adult action potentials, a large sodium influx causes depolarization. This is followed by an inward calcium flux, which is larger in the day 1 action potential. The calcium influx continues for longer in the immature action potential, contributing to a plateau phase during repolarization and there are two phases of outward potassium current, causing a two-stage repolarization. By comparison, in the adult action potential (FIG. 2C), there is a shorter influx of calcium due to more rapid calcium current deactivation, corresponding to no plateau and a shorter action potential duration (Guo, W., et al. (1996) The American Physiological Society—Cell Physiology 271(1):C93). There is also an increase in transient outward current, tied to increasing potassium selectivity in these ion channels, as well as more rapid deactivation of inwardly rectifying potassium currents. As a result, more rapid repolarization, as well as shorter action potential duration are observed (Kilborn, M., Fedida, D. (1990) Journal of Physiology 430:37-60).

Another electrophysiological measure of cardiomyocyte maturity is conduction velocity. As cardiomyocytes develop and mature, their conduction velocity increases (De Boer, T., et al. (2008) Netherlands Heart Journal 16(3):106-109; Thomas, S., et al. (2003) Circulation Research, 92:1209- 12 16).

Based on the above, three design criteria for a novel in vitro model of innervated myocardium were defined:

1) Co-cultures must have spatially organized tissue. The cardiomyocytes must be aligned in an anisotropic monolayer, and they should exhibit an elongated, rod-like morphology that parallels adult cardiomyocytes in vivo;

2) The co-culture microenvironment must be optimized. Using two-day old rat ventricular myocytes and cortical neurons, the media components and seeding conditions must be optimized to best embed the neural networks in the anisotropic muscle monolayers, and to engender a functional effect on the cardiomyocytes; and 3) Cardiomyocytes in co-culture must exhibit mature action potential properties. This includes rapid repolarization and short action potential duration, on the scale of in vivo action potential characteristics.

### Cardiomyocyte Isolation

Ventricular tissue was isolated from two-day old Sprague-Dawley rats (Charles River Laboratories) in accordance with the Institutional Animal Care and Use Committee guidelines at Harvard University. Ventricles were cut in 4-6 pieces and enzymatically digested in 0.1% trypsin (US B) solution for 14-16 hours at 4° C. Cardiomyocytes were then dissociated from the tissue by two minute incubation in 0.1% collagenase solution at 37° C. Myocytes were then centrifuged at 1200 rpm for 10 minutes, re-suspended in myocyte media (Table 1) and filtered using a 40 tm nylon cell strainer (BD Bioscience). Two 45-minute pre-plate steps (plating the cell suspension on a T75 or T175 flask, respectively) followed to filter out fibroblasts. Cells were then seeded on coverslips as described below and maintained in a 37° C., 5% carbon dioxide incubator (Thermo Electron Corporation). Myocyte media was changed on day 1, day 2 and then every 48 hours.

TABLE 1
A comparison of myocyte, neuron
and co-culture media components.

| Media Components | Myocyte | | Neuron | | Co-culture | |
|---|---|---|---|---|---|---|
| Medium 199 (Gibco | 430 | mL | | | 430 | mL |
| 11150-059) | | | | | | |
| MEM non-essential | 5 | mL | | | 5 | mL |
| (Gibco 15630-080) | | | | | | |
| DMEM (Gibco | | | 440 | mL | | |
| 11995-065) | | | | | | |
| Fetal Bovine Serum, | 50 | mL | 50 | mL | 50 | mL |
| Heat-Inactivated | | | | | | |
| (Gibco 16140-071) | | | | | | |
| L-Glutamine (Gibco | 2 | mM | 2 | mM | 2 | mM |
| 25030-081) | | | | | | |
| HEPES (Gibco | 5 | mL | | | 5 | mL |
| 15630-080) | | | | | | |
| Vitamin B12 (Sigma | 3 | mM | | | 2 | mM |
| V-2876) | | | | | | |
| Glucose | 20 | mM | 30 | mM | 20 | mM |
| p-Aminobenzoic | | | 7 | μM | | |
| Acid (Sigma A-3659) | | | | | | |
| KCl | | | 25.4 | mM | | |
| Insulin (Sigma I- | | | 5 | μg/mL | | |
| 1882) | | | | | | |
| Vitamin C (Sigma A- | 140 | mM | | | 140 | mM |
| 4544) | | | | | | |
| Penicillin (Sigma P- | 50,000 | U/mL | 100 | U/mL | 50,000 | U/mL |
| 4687) | | | | | | |
| Streptomycin | | | 100 | μg/mL | | |
| Epinephrine (Sigma | 5 | mM | | | 5 | mM |
| E-4250) | | | | | | |
| Nerve Growth Factor | | | 10 | μM | | |
| (Sigma N-6009) | | | | | | |

### Soft Lithography & Micropatterning

Soft lithography and micropatterning techniques were used to engineer the cardiomyocytes into spatially organized, anisotropic monolayers (FIG. 3). In soft lithography (FIG. 3A), a two-dimensional design in AutoCAD is transformed to a three-dimensional rubber stamp. The first step was to template the spatial organization that was desired for the tissue to have by designing a mask in AutoCAD. The goal was to align the tissue, and masks designed to have uniaxial lines with gaps in between were used. The line and gap dimensions that were used ranged from 6 pm to 30 μm; masks with 10 micron lines and 10 micron gaps were chosen for their success in aligning cardiomyocytes.

In the next step, negative photolithographic techniques were used to etch the pattern onto a silicon wafer. After this step, the wafer was a three-dimensional negative of the template for tissue organization with lines as valleys and ridges as gaps. An organic elastomer, polydimethylsiloxane, PDMS (Sylgard 184, Dow Corning), was then poured onto the etched wafer. After vacuum desiccation to remove surface impurities, the PDMS-coated wafer was baked at 65° C. overnight. The hardened, PDMS stamp which had the positive image of the template was peeled off, with its valleys as the gaps and ridges as the patterned lines.

In the next step, micropatterning techniques were used to “ink” the PDMS stamp and to stamp down a biological template for the cardiomyocyte organization (FIG. 3B). First, an extracellular matrix protein, e.g., fibronectin, at a concentration of 50 μg/mL was spread on a PDMS stamp and incubated for 1 hour. Extracellular matrix (ECM) proteins such as collagen, laminin and fibronectin are molecules that exist throughout the body and mechanically couple cells to their external environment. After incubation, the excess fibronectin was removed and pressed down the stamp onto a PDMS-coated, 25 mm glass coverslip. Due to the three-dimensional topography of the stamp, only the ECM-coated ridges touched the surface, transferring the ECM-protein to the coverslip in these areas, but not where the valleys were. Thus, the glass coverslip was micropatterned with the 10 micron line pattern, with 10 micron gaps in between.

The entire coverslip was then coated with a weaker concentration of fibronectin, 2.5 g/mL and incubated for 10 minutes. Thus, a fibronectin surface was prepared that could guide the myocytes along the high concentration lines while allowing other myocytes to slowly grow laterally along the low concentration gaps, creating a confluent anisotropic monolayer.

The micropatterned coverslips were seeded with either 1 million or 1.5 million myocyte cells, isolated as described above.

### Immunofluorescent Microscopy

Immunofluorescent microscopy was used to evaluate cellular adhesion to the micropattern as well as individual cellular architecture. To immunostain, cultures were fixed in 4% parafonnaldehyde (Electron Microscopy Sciences #15710) with Triton X-100 (Sigma X1001 L) for 15 minutes and stained against DAPI (Invitrogen), sarcomeric α-actinin (Sigma, clone EA-53) and/or connexin 43 (Sigma C-6319). Immunostained cultures on microscope slides were mounted and obtained immunofluorescent images using a Leica DM1 6000b microscope.

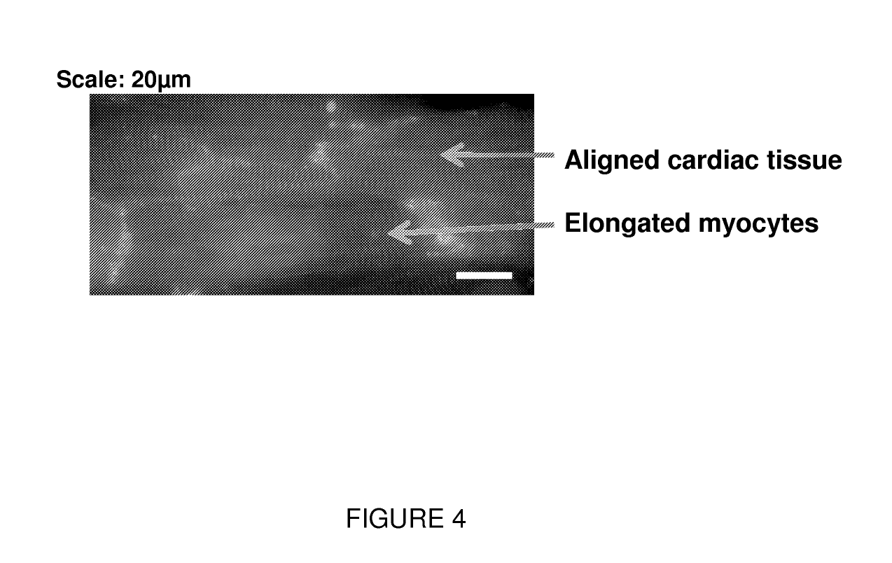

Immunofluorescent microscopy confirms that anisotropic cardiomyocyte monolayers were generated using the soft lithographic and micropatterning techniques detailed above. The cardiomyocytes readily adhered to the fibronectin surface and spatially organized according to the tissue template. FIG. 4 shows an immunostained image of the engineered cardiac monolayer. The widespread sarcomeric cc-actinin fluorescence in FIG. 4 indicates the confluence of the monolayer. Furthermore, the sarcomeres have adopted a uniform, vertical alignment. Sarcomeres orient perpindicular to the length-wise direction of force; thus, uniform, vertical sarcomere alignment indicates successful tissue alignment, with a horizontal axis of anisotropy. In FIG. 4, the tissue orientation is indicated by the white line, perpendicular to the series of vertical, fluorescent sarcomeres. Immunostaining also confirmed elongated individual cell architecture. These results demonstrate that cardiomyocyte cultures can be engineered in vitro to form spatially organized anisotropic monolayers with in vivo-like cell morphology.

### Example 2

### Co-Culture Optimization

Optimal co-culture conditions were determined for embedding neural networks in the engineered cardiac monolayers produced as described above.

Maintaining the health and functionality of two different cell types, ventricular cardiomyocytes and cortical neurons, together in co-culture inherently places several design constraints upon an in vitro model. The cellular microenvironment needs to be optimized for each cell type. The parameter space included finding the optimal co-culture media and determining cell seeding order which would best embed the neurons. Adapting the extracellular matrix micropatterning protein to the co-culture situation and optimizing cell seeding density was also considered.

Two different neuron isolation methods were explored. Briefly, cortices were isolated in parallel with ventricular myocytes, and the tissue was trypsinized overnight as in the myocyte isolation described above. The brain tissue was then homogenized, filtered and re-suspended similar to that described in the myocyte isolation. The neural suspension at this point contained neurons as well as several other, undesired cell types including glial cells such as astrocytes and oligodendrocytes as well as fibroblasts and endothelial cells. These cells, although present in the in vivo heart and possibly implicated in myocardial function, do not contribute to cardiac signal propagation and contraction. Thus, in a controlled microenvironment, these cells represent an unnecessary variable. To attempt to filter out these undesired cells, one of two methods was used.

The first pre-plate method filtered the neural suspension by pre-plating it on a T175 flask for 2 hours. As glial cells and fibroblasts adhere more rapidly to surfaces, the unattached cells were kept in hopes of a more pure neuron sub-population. The second pre-plate method used a negative filter of the neural suspension. A flask was coated with 0.0 1% Poly-L Lysine, PLL (Sigma P-4707), a charged particle that neurons and few other cells will adhere to. The neural suspension was pre-plated on these PLL-coated flasks for 24 hours; unattached cells were aspirated and attached cells, an enriched neuron sub-population, were trypsinized in 0.1% trypsin at 37° C. for 15 minutes and re-suspended. These neuron-enriched suspensions were used to seed on the cardiomyocytes.

The measures for optimization of the co-culture method were minimized cell death (apoptosis or necrosis) and maximized cellular adhesion and micropattemed line coverage.

The first challenge was determining a single cell media that would allow the seemingly disparate cell types to grow together in an appropriate microenvironment. The media components of myocyte media alone with those of neuron media alone were compared. There were some identical components such as fetal bovine serum, L-glutamine, and penicillin as well as similarities between their two base compounds, Minimum Earle's Medium 199 (MEM 199) and Dulbecco's Modified Earle's Medium (DMEM). This simplified the problem by a few components.

Next, it was determined if any components of either cell's media were particularly helpful or harmful to the other cell type. Two different ideas emerged. For one, other in vitro co-culture studies of neurons and cardiomyocytes have diverse media recipes, but many share one component: nerve growth factor, NGF, a signaling factor shown to promote neuronal survival. This indicated that NGF was an important factor. Secondly, the neuron media has both penicillin and streptomycin antibiotics, while the myocyte media has only penicillin. Streptomycin is known to block stretch-activated ion channels, thus, the myocyte media without streptomycin was determined to be the more versatile media.

Based on the results, it was discovered that neurons could grow in myocyte media, as it shares many of the same components. This offered ease, as all the components were already available, and lacked the potentially undesired neuron media ingredient, streptomycin. NGF was added to the myocyte media to promote neuron survivability in co-culture.

This solution allows both cardiomyocytes and neurons to adhere and survive in co-culture for 1-3 weeks.

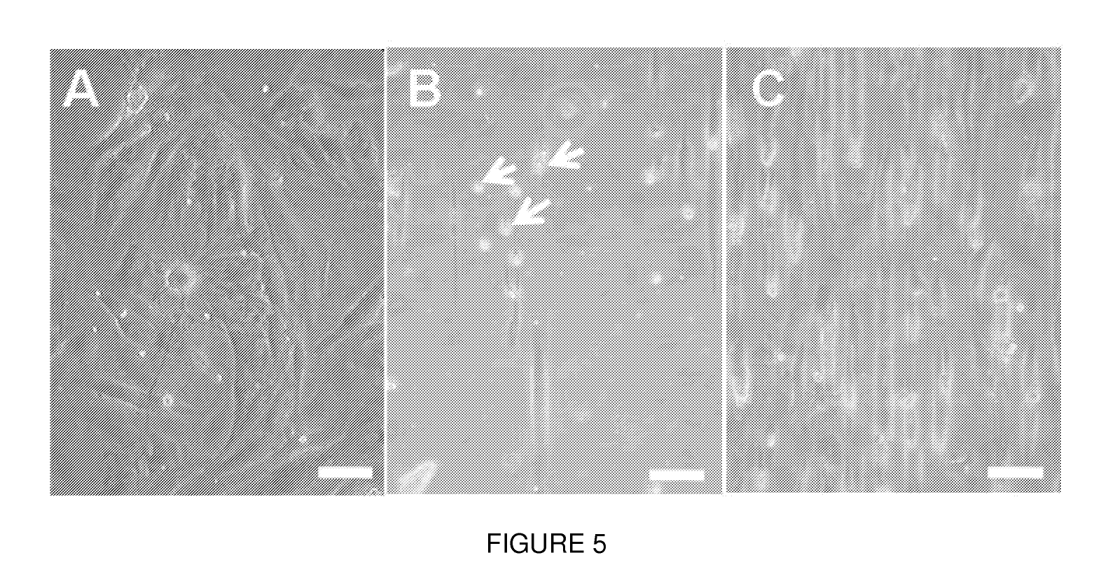

Next, the seeding order and timing were considered. Seeding neurons 7 days or 2 hours before the myocytes, or 2 hours, 24 hours or 3 days after the myocytes was explored. It was found that the myocytes must be seeded first to best embed the neural networks and maintain micropatterned coverage. These results are shown in FIG. 5. Seeding neurons 7 days before the myocytes produced poor pattern coverage (FIG. 5A). Neural suspensions have cell types other than neurons including many fibroblasts. As fibroblasts are the primary source of extracellular matrix in the heart, it is likely that these cells over the course of the seven days laid down their own extracellular matrix proteins, in a pattern other than the template provided. Seeding the neurons 2 hours before the myocytes restricted adhesion of both cell types, and seemed to increase cell death (FIG. 5B) as seen by a large number of round, floating cells in culture.

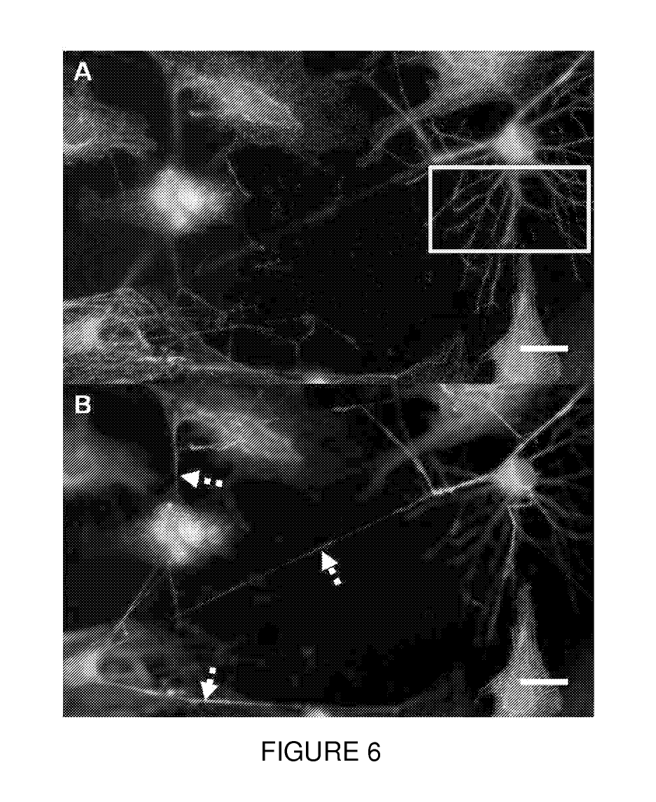

By contrast, co-cultures seeded with myocytes 2 hours (FIG. 5C), 24 hours, or 3 days before the neurons, exhibited good cell adhesion and good patterned line coverage. It was noticed that although neurons would adhere to the substrate in the absence of other options, the connections between them would often adopt a higher z-plane (FIG. 6). The reason lies in each cell type's substrate stiffness preference. Cardiomyocytes prefer to adhere to stiff substrates, from which they can contract with more force, while neurons prefer softer substrates like those found inside of the brain. The Young's modulus of the PDMS-coated glass coverslip is approximately 1.5 MPa, while the cardiomyocytes themselves have a Young's modulus of around 30 kPa. Thus, the cardiomyocytes find a stiff substrate when seeded first onto the PDMS-coated glass coverslip, while the neurons find the soft substrate they desire with the cardiomyocytes themselves.

This simplified the search for the best extracellular matrix protein to use to template the tissue organization. Since the optimized seeding order required seeding myocytes on the glass coverslip first, the template that was developed for myocyte cultures alone transferred to the co-culture situation with no alterations. Fiibronectin was chosen as a preferred micropattenning protein for the spatially organized co-culture.

The cell seeding concentrations were next optimized. Seeding cardiomyocytes using 2.5×10⁵, 1×10⁶, and 1.5×10⁶ cells was tested; it was found that 1.5 million cardiomyocytes ensured uniform cellular adhesion.

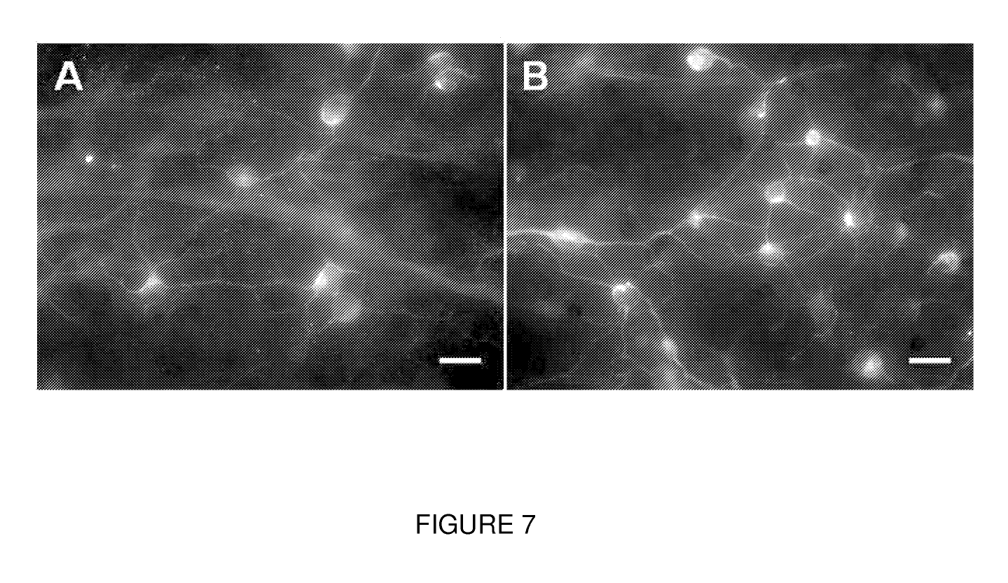

For the neuron seeding concentration, one of two different concentrations was used to evaluate the effect of neuron concentration on myocyte function. A low concentration of 3×10⁵ or a high concentration of 1.5×10⁶ neurons was used (FIG. 7). Lower concentrations of 5×10⁴ and 1×10⁵ neurons were also tried, but it was found that these concentrations were so low that the neurons could not reach each other to form neural networks. The low and high seeding concentration of neurons was kept as a variable in the model.

Finally, neuron-enriched suspensions from one of the two neuron isolation methods described previously were used to embed on the cardiomyocytes. The 2 hour and 24 hour pre-plate methods were kept as a second variable in the model.

With a reduced co-culture parameter space, the in vitro model maintained two variables: neuron seeding concentration and neuron pre-plate method. All following co-culture design results centered about one of these four co-cultures:

1) low seeding concentration, 2 hour pre-plate or “LO 2 h”;

2) high seeding concentration, 2 hour pre-plate or “HI 2 h”;

3) low seeding concentration, 24 hour pre-plate or “LO 24 h”; and

4) high seeding concentration, 24 hour pre-plate or “HI 24 h”.

Micropatterned coverslips were seeded with cardiomyocytes, followed by neuron seeding according to these co-culture specifications. Media for all co-cultures was changed on day 1, day 2, and then every 48 hours, to maintain an enriched cellular microenvironment. The next step was to characterize each of the co-culture populations.

### Characterization of Co-Culture Population

Before the effect of the embedded neural networks on myocyte function could be examined, the co-culture population needed to be characterized. It was hypothesized that lower neuron seeding concentration would correlate to a lower neuron population compared to the high seeding concentration. It was further hypothesized that the 24 hour pre-plate method would more effectively filter out the other, undesired cells (mainly glial cells and fibroblasts, as described in the neuron isolation approach), leaving a more pure population of neurons and myocytes.

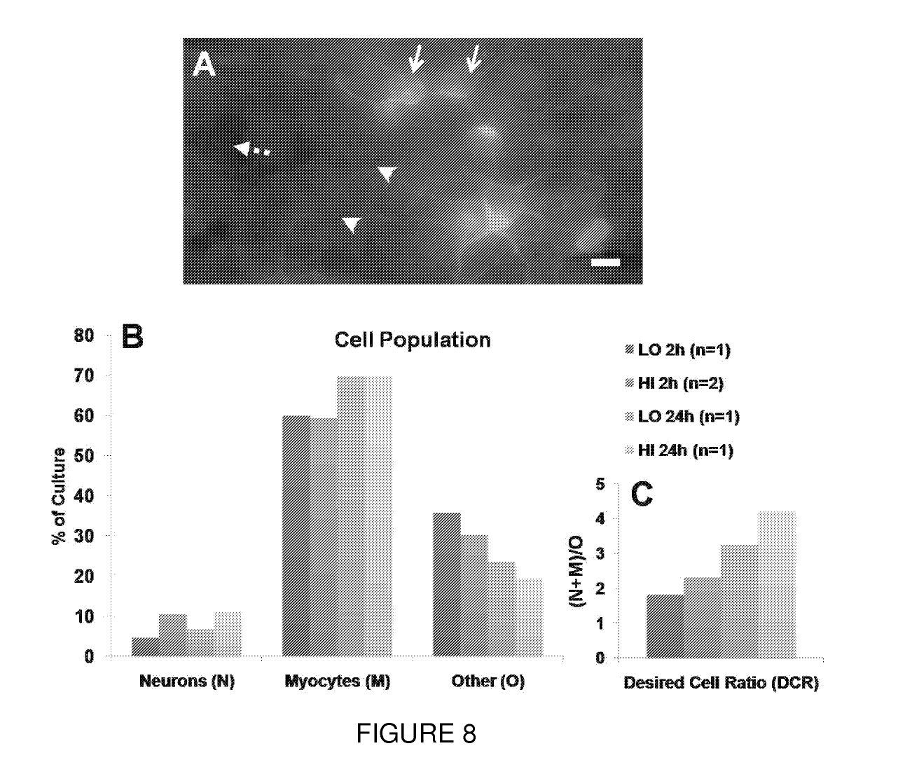

To confirm these hypotheses, immunofluorescent microscopy was used to characterize each co culture population. As described above, immunoflourescent staining was preformed against sarcomeric cc-actinin and DAPI. Staining against β-tubulin III, a neuron-specific marker was also performed. For each co-culture population, ten fields of view were imaged and each nucleus was characterized as belonging to a neuron, myocyte, or other cell type. These results are summarized in FIG. 8.

It was found that co-cultures with low neuron seeding concentrations had about 5-7% neurons (FIG. 8B), while those with high neuron seeding concentrations had a 10% neuron population. The 24 h co-cultures had the smallest percentages of other cells with 24% (LO 24 h) and 20% (HI 24h) populations instead of the 30% and 35% of the corresponding 2 h co-cultures. This explains why the 24 hour pre-plate co-cultures had a larger myocyte population, with ˜70% versus the 2 hour pre-plate co-cultures' 60% myocyte population. Although all co-cultures were seeded with 1.5 million myocytes, larger percentages of glial cells and fibroblasts in 2 h co-cultures competed with myocytes for space; fewer of these cells in 24 h co-cultures permitted more myocytes to adhere and survive.

The purity of the co-culture increased in parallel with the neuron seeding concentration, and with the 24 hour pre-plate method (FIG. 8C). The LO 2 h co-culture had the smallest desired cell ratio, DCR—the ratio of neurons and myocytes to undesired, other cells—of less than 2. The HI 24h co-culture had the largest DCR of greater than 4, representing the most pure population of neurons and cardiomyocytes.

### Characterization of Electrophysiology

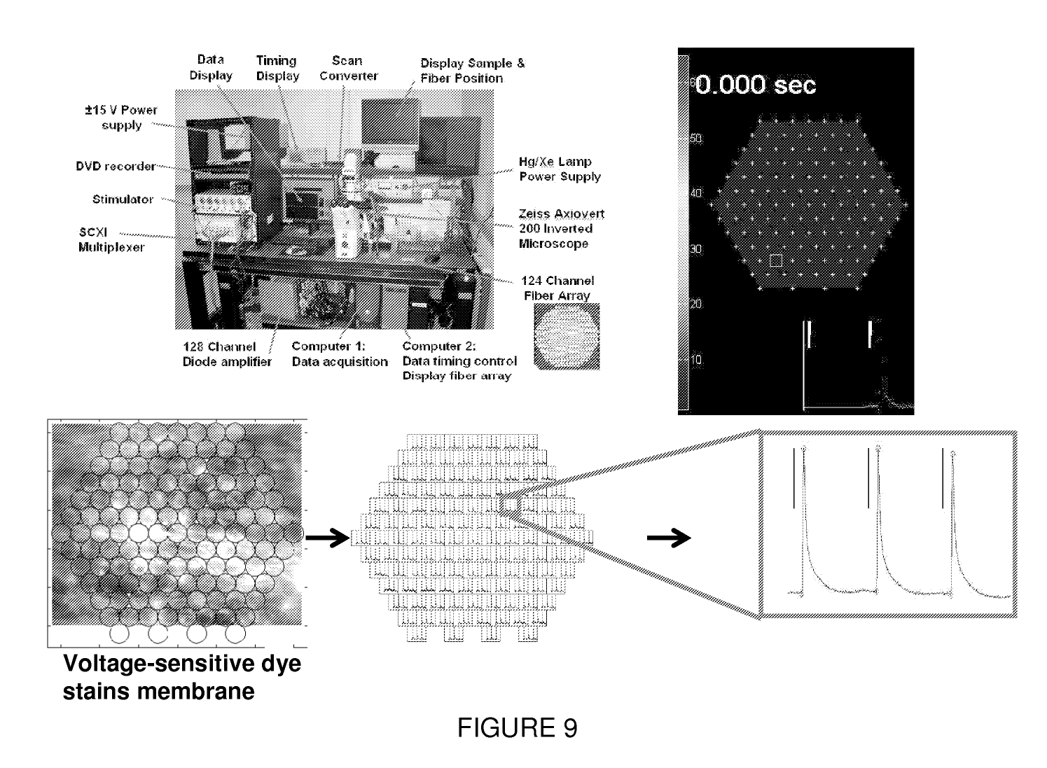

To characterize the cardiomyocyte electrophysiology in co-culture with neurons, an optical mapping system as shown in FIG. 9 was used. The tool employs a 124 photodiode-coupled optical fiber array and is useful for voltage mapping recordings and was used to record action potentials.

Day 4 co-cultures were transferred to the platform of a Zeiss Axiovert 200 Inverted Microscope, maintained in Tyrode's solution at 37° C. with a heated bath (BioScience Tools). They were stained for 5 minutes with 8 μM of the voltage-sensitive dye, RH237 (Invitrogen S-1109). They were then treated with 10 uM Blebbistatin (Calbiochem 203390), an excitation-contraction decoupler to remove motion artifact. Co-cultures were then point stimulated with 6-10 V at 2 Hz and fluorescent recordings were taken from a field of view a distance away. The optical fibers of the optical mapping system can interpret the subtle changes in cardiomyocyte membrane fluorescence, directly proportional to the changes in transmembrane potential, the action potential. Action potentials from one or more coverslips of each co-culture on day 4, across four or more fields of view per coverslip were recorded. Action potentials from control coverslips prepared and treated just as co-culture coverslips, but seeded with myocytes only were also recorded. The resulting data had spatial and temporal resolution.

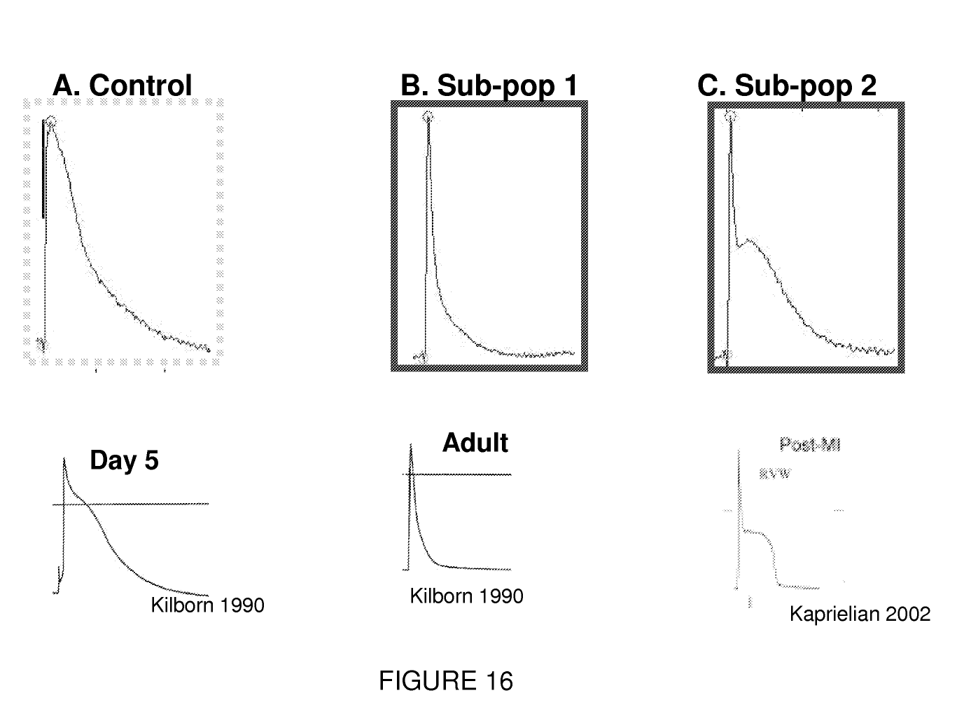

The recorded cardiomyocyte action potentials in co-culture expressed morphological and quantitative differences compared to myocyte only controls. Two distinct co-culture action potential morphologies were observed; each one having a rapid repolarization and one with a short action potential duration. See, e.g., FIG. 16.

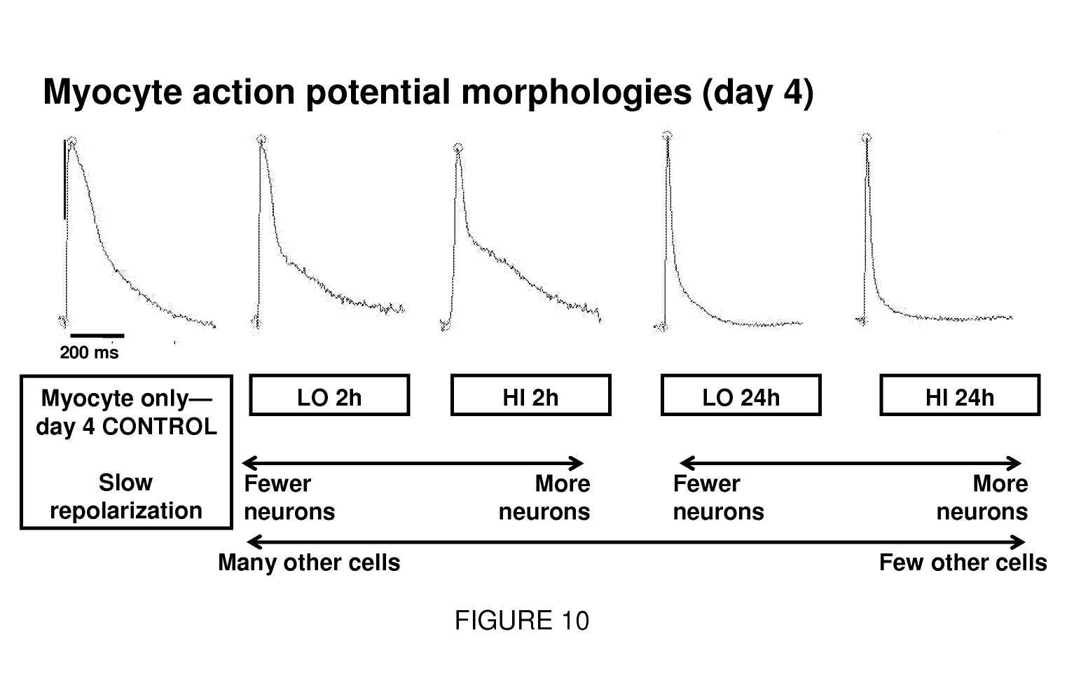

In addition, the cardiomyocyte action potential morphology in each of the day 4 co-cultures had a more mature phenotype in comparison to its day 4 control culture counterpart (FIGS. 10 and 16). The control looked as might be expected of a day 4 cardiomyocyte action potential, with slow repolarization and long action potential duration. In comparison, each co-culture action potential exhibited rapid repolarization. This corresponds to a steep slope after peak depolarization or a narrower, more pointed peak in comparison to the broad, triangle-like peak characteristic of an immature cardiomyocyte action potential. The overall duration of the action potential seemed to decrease with increased co-culture purity; the 24 h co-cultures with the fewest numbers of other cells had particularly short action potential durations. The cardiomyocyte action potential morphologies in co-culture, with rapid repolarization and short action potential duration, thus satisfied the rubrics for increased electrophysiological maturity of the cardiomyocytes.

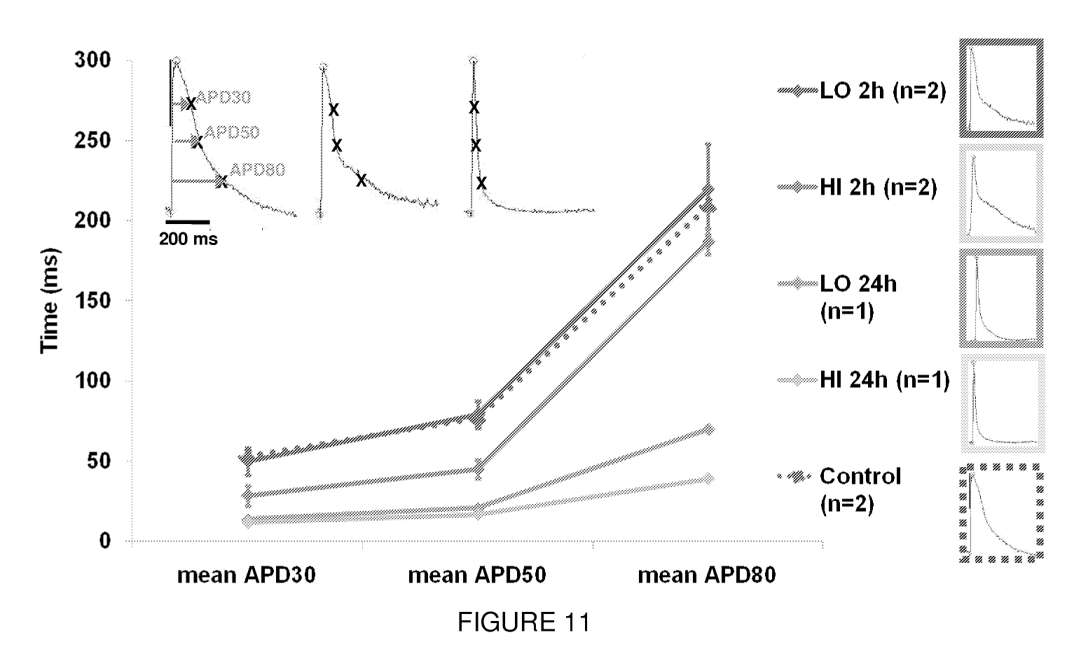

In order to quantify the profound qualitative differences in action potential morphology, the time it took after activation for the action potential to peak and fall 30% of the maximum amplitude was measured. This is referred to as action potential duration 30 or APD30. Similarly, the time to reach 50% and 80% of the maximum amplitude was measured (referred to as APD50 and APD80, respectively). These values are plotted for each of the co-culture conditions in FIG. 11.

These quantitative results confirm more mature action potential phenotypes in all of the co-cultures. All of the co-cultures had smaller APD30's than the control myocyte, indicating more rapid repolarization in all four co-cultures. The HI 2 h co-culture had a smaller APD30, APD50, and APD80 compared to the control myocyte, although the differences were not as stark as either of the 24 h co-cultures. In the LO 24 h and HI 24 h co-cultures, the APD30's, 50's and 80's were at most 25% the magnitude of the corresponding control myocyte values. In particular, the overall action potential durations in these two co-cultures is an order of magnitude smaller than the control myocyte with APD80's of 69.7 ms (LO 24 h) and 38.9 ms (HI 24 h) compared to 208.8 ms (control). The HI 24 h co-culture exhibited the most rapid repolarization, indicated by the smallest APD30 and APD50, and shortest duration, indicated by APD80. The HI 24 h APD30, 50 and 80 values of 11.8, 16.7 and 38.9 ms are much smaller than the control myocyte values of 52.6, 72.6 and 208.8 ms.

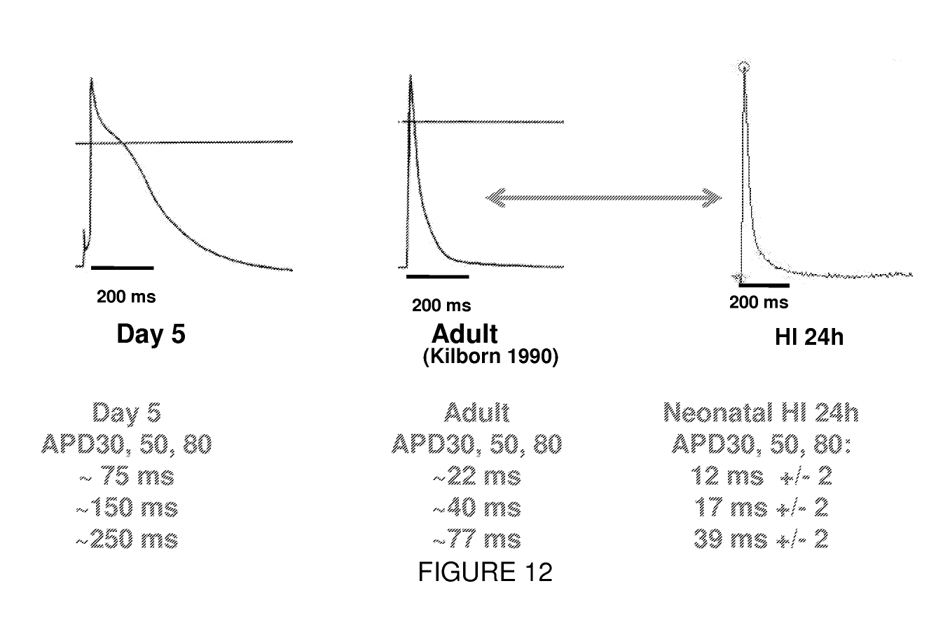

The HI 24 h co-culture cardiomyocyte electrophysiology bears much more resemblance to analogous values of an adult myocyte than a more immature myocyte. A quantitative comparison of these APD30, 50 and 80 values follows in FIG. 12, accompanied by their corresponding morphologies. The APD's of the day 4 control are largely similar to values determined at day 5 during development—in particular, the APD80 values of 211 ms and 250 ms, respectively; meanwhile, the APD values for day 4 cardiomyocytes in the HI 24 h co-culture are more similar to adult cardiomyocyte APD's in development with small APD80 values of 39 ms and 77 ms, respectively. From a morphological perspective, the similarity between the mature myocyte action potential and our HI 24 h co-culture action potential is striking.

In summary, the above experiments demonstrate that any of the four co-cultures may achieve more mature cardiomyocyte action potentials with the HI 24 h co-culture exhibiting mature, adult-like action potentials.

It was next determined if the changed action potentials were due to neural paracrine signaling factors, the chemical messengers that allow remote cells to communicate. Previous work suggested that cardiomyocyte contractile properties were matured by the presence of conditioned media, media enriched with chemical messengers between cells (Lloyd, T., Marvin Jr., W. (1989) Journal of Molecular and Cellular Cardiology 22:333-342). In contrast, it is hypothesized herein that contact between neurons and myocytes is critical for accelerating cardiomyocyte electrophysiological maturation. It should also be noted that the previous study did not use cortical neurons, which do not secrete acetylcholine, epinephrine, and norepinephrine, paracrine signaling factors known to affect cardiomyocyte function.

Briefly, neuron media was conditioned in neural cultures for 24-30 hours to enrich the media with neural paracrine signaling factors. The media was then filtered through a 40 μm nylon cell strainer (BD Bioscience) and stored at −20° C. Conditioned media was thawed and applied to day 3 myocyte only cultures, prepared as described in Lloyd (supra), for 48 hours. The conditioned cardiomyocyte cultures were optically mapped on day 5 as described above.

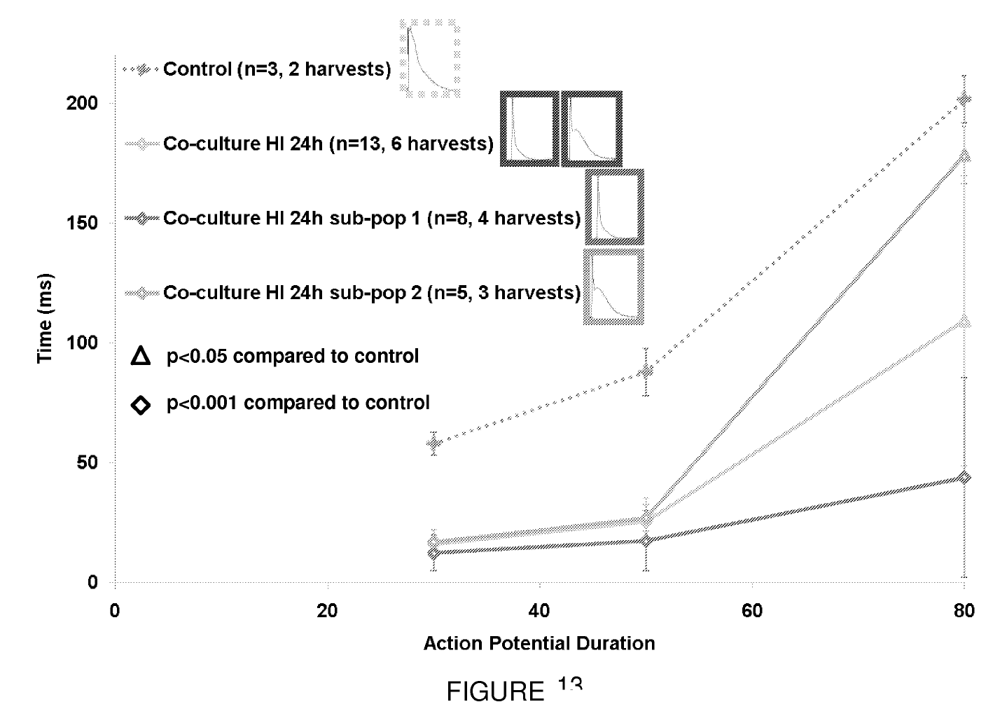

The difference between the action potential durations was not striking (FIG. 13). The conditioned media myocytes had slightly smaller APD30, 50 and 80′s of 63.0, 101.4 and 257.0 ms compared to day 5 myocyte control values of 79.2, 122.4, and 270.9. Overall, however, the action potential morphology and durations reflected immature myocyte electrophysiology, with slow repolarization and long action potential duration. These results indicate that neural paracrine signaling does not play a primary role in the accelerated maturation of the co-cultures. Rather, the results demonstrate that direct contact between neurons and cardiomyocytes is important for accelerating cellular maturation.

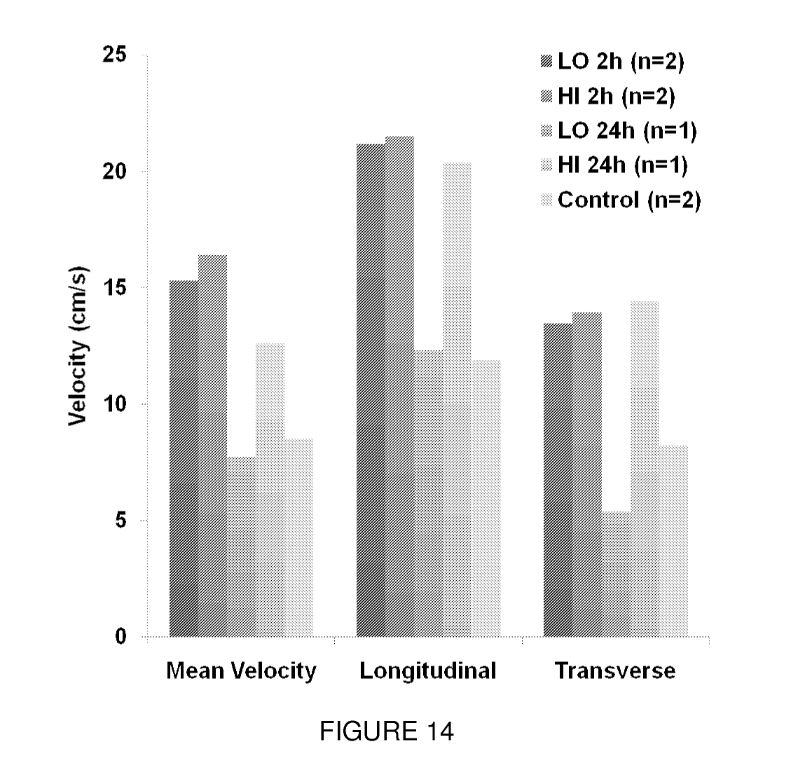

Another measure of cardiomyocyte electrophysiological maturation, conduction velocity, was assayed. Using the spatial and temporal data provided by the optical mapping system, the overall conduction velocity across several fields of view from each co-culture coverslip was calculated. Conduction velocity longitudinal and transverse to the axis of anisotropic orientation was also calculated. These results are summarized in FIG. 14.

No clear trends were noticed indicating that conduction velocity might increase proportional to the increase in action potential maturity. Conduction velocity increases with cardiomyocyte maturity, thus it might be expected that there are larger conduction velocities in the co-cultures as compared to controls, particularly in the HI 24 h co-culture. However, the results presented herein demonstrate that the increase in conduction velocity that occurs in parallel with increased cardiomyocyte maturity is primarily attributed to an increase in cell size which occurs during development. It is not likely that neural cells prompt increasing cardiomyocyte cell volume in co-culture. Although these results have shown electrophysiological maturity of cardiomyocyte action potential properties in co-culture, it has been shown that there is merely a minimal co-culture effect on conduction velocity.

### Candidate Drug Testing

Co-cultures were prepared as described herein and the effect of ion channel blockers, e.g., Na⁺ channel blockers, e.g., Tetrotodoxin, Ca²⁺ channel blockers, e.g., Nifedipine, were tested on the action potential of the cells using the apparatus shown in FIG. 9. The predicted and actual results of these experiments are shown in Table 2 confirming that it is the action potential of the innervated myocytes that is being optically mapped.

TABLE 2
Effect of ion channel blockers on the action potentials of co-cultured cells.

| | | | | | | |
|---|---|---|---|---|---|---|
| | | | | Observed | Observed | |
| | | Expected | Expected | effect on | effect on | Observed |
| | Channel | effect on | effect on | Co-culture | Co-culture | effect on |
| Ion channel | Blocker | neonatal AP | mature AP | Sub-pop 1 | Sub-pop 2 | Control |

| | | | | | |
|---|---|---|---|---|---|
| Na+ | TTX | little to none | AP inhibition | little to none | little to none |
| L-type Ca2+ | Nifedipine | AP inhibition | little to none | little to none | AP inhibition |

Equivalents

In describing embodiments of the invention, specific terminology is used for the sake of clarity. For purposes of description, each specific term is intended to at least include all technical and functional equivalents that operate in a similar manner to accomplish a similar purpose. Additionally, in some instances where a particular embodiment of the invention includes a plurality of system elements or method steps, those elements or steps may be replaced with a single element or step; likewise, a single element or step may be replaced with a plurality of elements or steps that serve the same purpose. Further, where parameters for various properties are specified herein for embodiments of the invention, those parameters can be adjusted up or down by 1/20th, 1/10th, ⅕th, ⅓rd, ½, etc., or by rounded-off approximations thereof, unless otherwise specified. Moreover, while this invention has been shown and described with references to particular embodiments thereof, those skilled in the art will understand that various substitutions and alterations in form and details may be made therein without departing from the scope of the invention; further still, other aspects, functions and advantages are also within the scope of the invention. The contents of all references, including patents and patent applications, cited throughout this application are hereby incorporated by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

1. A method for preparing an engineered innervated tissue, comprising
providing a solid support structure comprising a patterned biopolymer;
seeding immature cells on the patterned biopolymer;
culturing the cells such that an anisotropic tissue forms;
seeding the anisotropic tissue with neurons; and
culturing the anisotropic tissue seeded with the neurons to form an anisotropic tissue with embedded neural networks, thereby preparing an engineered innervated tissue.

2. A method for accelerating maturation of a cultured cell, comprising
providing a solid support structure comprising a patterned biopolymer;
seeding immature cells on the patterned biopolymer;
culturing the cells such that an anisotropic tissue forms;
seeding the anisotropic tissue with neurons; and
culturing the anisotropic tissue seeded with the neurons to form an anisotropic tissue with embedded neural networks, thereby accelerating maturation of a cultured cell.

3.-11. (canceled)

12. The method of claim 1 or 2, wherein the solid support structure further comprises a sacrificial polymer layer and a transitional polymer layer.

13. The method of claim 12, wherein the sacrificial polymer is a degradable biopolymer.

14. (canceled)

15. The method of claim 1 or 2, wherein the immature cells are myocytes.

16.-18. (canceled)

19. The method of claim 1 or 2, wherein the neuron is a neuron that does not secrete acetylcholine, epinephrine and/or norepinephrine.

20. The method of claim 1 or 2, wherein the neurons are cortical neurons

21. The method of claim 1 or 2, wherein the neurons are seeded at a density of at least about 1.5×10⁶ per millimeter

22. The method of claim 15, wherein the myocytes are cultured for about 24 hours prior to the seeding of the neurons.

23. The method of claim 15, wherein the myocytes are cultured for a period selected from the group consisting of about 2, 4, 6, 8, 10, 12, 14, 16, 18, 20, 22, 24, 26, 28, 30, 32, 34, 36, and 48 hours prior to the seeding of the neurons.

24.-28. (canceled)

29. A method for assaying a biological activity, comprising:
providing an engineered innervated tissue prepared according to the method of claim 1; and
evaluating an activity of the tissue, thereby assaying a biological activity.

30. The method of claim 29, wherein evaluating a biological activity comprises evaluating the contractility of a cell, the mechano-electrical coupling of a cell, the mechano-chemical coupling of a cell, and/or the response of a cell to varying degrees of substrate rigidity.

31. A method for identifying a compound that modulates a tissue function, the method comprising
providing an engineered innervated tissue prepared according to the method of claim 1;
contacting said tissue with a test compound; and
determining the effect of the test compound on a tissue function in the presence and absence of the test compound, wherein a modulation of the tissue function in the presence of said test compound as compared to the tissue function in the absence of said test compound indicates that said test compound modulates a tissue function, thereby identifying a compound that modulates a tissue function.

32. A method for identifying a compound useful for treating or preventing a tissue disease, the method comprising
providing an engineered innervated tissue prepared according to the method of claim 1;
contacting said tissue with a test compound; and
determining the effect of the test compound on a tissue function in the presence and absence of the test compound, wherein a modulation of the tissue function in the presence of said test compound as compared to the tissue function in the absence of said test compound indicates that said test compound modulates a tissue function, thereby identifying a compound useful for treating or preventing a tissue disease.

33.-36. (canceled)

37. A method of fabricating a pacemaker, comprising
providing a base layer;
coating a sacrificial polymer layer on the base layer;
coating a flexible polymer layer that is more flexible than the base layer on the sacrificial polymer layer;
seeding cells on the flexible polymer layer;
culturing the cells such that an anisotropic tissue forms;
seeding the anisotropic tissue with neurons;
culturing the anisotropic tissue seeded with the neurons to form an anisotropic tissue with embedded neural networks; and
releasing the flexible polymer layer from the base layer to produce a pacemaker graft comprising the tissue structure, wherein the tissue structure is configured for epicardial attachment and is further configured to propagate an action potential through the attached tissue.

38.-41. (canceled)

42. A method of treating a subject with a bradyarrythmia, comprising attaching the pacemaker prepared according to the method of claim 37 to the epicardium of the subject, thereby treating the subject with a bradyarrythmia.

43. A method of treating a subject with an AV-node conduction defect, comprising attaching the pacemaker prepared according to the method of claim 37 to the epicardium of the subject, such that the AV-node is bypassed.
